(19) United States
(12) Patent Application Publication
(10) Pub. No.: US 2025/0074920 A1
(43) Pub. Date: Mar. 6, 2025

(54) MACROCYCLIC COMPOUNDS AND USE AS KINASE INHIBITORS

(71) Applicant: BLOSSOMHILL THERAPEUTICS, INC., San Diego, CA (US)

(72) Inventors: Jingrong Jean CUI, San Diego, CA (US); Eugene Yuanjin RUI, San Diego, CA (US); Evan W. ROGERS, San Diego, CA (US); Dayong ZHAI, San Diego, CA (US)

(21) Appl. No.: 18/725,951

(22) PCT Filed: Jan. 3, 2023
(86) PCT No.: PCT/US2023/060039

§ 371 Date: Jul. 1, 2024

Related U.S. Application Data
(60) Provisional application No. 63/296,705, filed on Jan. 5, 2022.
(60) Provisional application No. 63/435,654, filed on Dec. 28, 2022.

(51) Int. Cl. C07D 498/22; A61K 31/4162; A61K 31/4188

(52) U.S. Cl. CPC C07D 498/22; A61K 31/4162; A61K 31/4188

(57) ABSTRACT

The present disclosure relates to macrocyclic compounds, pharmaceutical compositions containing macrocyclic compounds, and methods of using macrocyclic compounds to treat disease, such as autoimmune disease.

### CROSS-REFERENCE TO RELATED APPLICATIONS

This application claims priority under 35 U.S.C. § 119(e) to U.S. Provisional Application Ser. No. 63/296,705 filed on Jan. 5, 2022 and U.S. Provisional Application Ser. No. 63/435,654 filed on Dec. 28, 2022, the entire disclosures of both of which are incorporated herein by reference.

### TECHNICAL FIELD

The present disclosure relates to macrocyclic compounds, pharmaceutical compositions containing macrocyclic compounds, and methods of using macrocyclic compounds to treat disease, such as human autoimmune diseases.

### BACKGROUND

Protein kinases are tightly regulated signaling proteins that orchestrate the activation of signaling cascades by phosphorylating target proteins in response to extracellular and intracellular stimuli. The human genome encodes approximately 518 protein kinases (Manning G, et al The protein kinase complement of the human genome. Science. 2002, 298:1912-34). Dysregulation of kinase activity is associated with many diseases, including autoimmune diseases, and cardiovascular, degenerative, immunological, infectious, inflammatory, and metabolic diseases (Levitzki, A. Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et al New Perspectives, Opportunities, and Challenges in Exploring the Human Protein Kinome. Autoimmune disease Res. 2018, 78:15-29). The critical role of kinases in autoimmune disease and other diseases makes them attractive targets for drug inventions with 52 small molecule kinase inhibitors have been approved and 46 of them for autoimmune disease targeted therapies (Roskoski R Jr, Properties of FDA-approved Small Molecule Protein Kinase Inhibitors: A 2020 Update. Pharmacol Res 2020, 152:104609). Cytokine signaling is essential for cell growth, hematopoiesis, and immune system function. Cytokine-mediated receptor dimerization induces intracellular activation of receptor-bound Janus kinases (JAKs), which then induce downstream transcriptional responses. The Janus Kinase Signal Transducer and Activator of Transcription (JAK-STAT) pathway plays a significant role in both normal and pathological states of immune-mediated inflammatory diseases (O'Shea J J, et al The JAK-STAT pathway: impact on human disease and therapeutic intervention. Annu Rev Med. 2015, 66:311-28). Targeting JAK-associated pathways by JAK inhibitors has achieved clinical success for a wide array of diseases, including ruxolitinib and fedratinib for myeloproliferative neoplasms, and tofacitinib, upadacitinib, and baricitinib for rheumatoid arthritis and other immune-mediated inflammatory disease (McLornan D P, et al Lancet. 2021, 398:803-816). However, it is challenging to achieve isoform selective JAK inhibitors and almost all these approved kinase domain ATP competitive JAK inhibitors display significant undesirable adverse effects due to inhibition of multiple JAK family members. The JAK kinases are large multidomain protein including the FER domain [JH6-JH7] and the SH2 domain [JH3-JH5], both mediating receptor interactions, the pseudokinase domain [JH2] with regulatory function, and the kinase catalytic domain [JH1](Garrido-Trigo A and Salas Journal A, Journal of Crohn's and Colitis, 2020, 5713-5724). The pseudokinase domain regulates the kinase domain by steric inhibition of ATP binding and/or a reduction in flexibility of the kinase active site required for catalysis (Patrick J, et al PNAS 2014 111: 8025-8030). Although JAK family members have high sequence homology within the catalytic domains, the distinguishing pseudokinase domain (JH2) in the JAK family could provide an ideal “allosteric” site for the development of highly selective JAK inhibitors. TYK2, a member of JAK family, play important role in regulating the signaling of a wide range of proinflammatory cytokines including IL12, IL23, and type 1 interferons (IFNα). A highly selective TYK2 inhibitor is needed for an optimal benefit-safety balance for the treatment of human autoimmune diseases including multiple sclerosis, Crohn's disease, psoriasis, etc (Leitner, N. R. et al Tyrosine kinase 2-surveillant of tumours and bona fide oncogene. Cytokine 2017, 89, 209-218).

Therefore, the discovery and development of highly selective inhibitors of the JAK family, such as TYK2 inhibitors targeting the TYK2 JH2 pseudokinase domain represents a new therapeutic invention for the treatment of human autoimmune diseases including multiple sclerosis, Crohn's disease, psoriasis, and the like.

### SUMMARY

In one aspect, the disclosure relates to a compound of the formula I, or a pharmaceutically acceptable salt thereof,

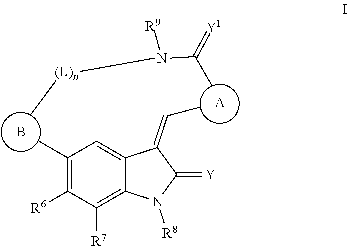

wherein
A is

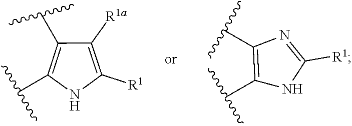

B is

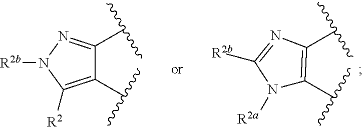

each L is independently —C(R³)(R⁴)—, —C(O)—, —O—, —N(R⁵)—, —S—, —S(O)— or —S(O)₂—, provided that (L)ₙ does not comprise a —O—O—, a —O—S—, a —S—S—, or a —O—N(R⁵)— bond, and (L)ₙ-N(R⁹)— does not comprise a —O—N(R⁹)— a —S—N(R⁹)— bond;
Y and Y¹ are each independently O or S;
each of R¹, R^{1a}, and R² is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R^{2a}, R⁵, R⁸, and R⁹ is independently H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R^{2b} is C₁-C₄ alkyl, C₂-C₄ alkenyl, C₂-C₄ alkynyl, C₃-C₄ cycloalkyl, or 3- to 4-membered heterocycloalkyl, wherein each hydrogen atom in C₁-C₄ alkyl, C₂-C₄ alkenyl, C₂-C₄ alkynyl, C₃-C₄ cycloalkyl, and 3- to 4-membered heterocycloalkyl is independently optionally substituted by deuterium, halogen, —O(H or C₁-C₂ alkyl), —OC(O)C₁-C₂ alkyl, —OC(O)N(H or C₁-C₂ alkyl)₂, —OS(O)C₁-C₂ alkyl, —OS(O)₂C₁-C₂ alkyl, —OS(O)N(H or C₁-C₂ alkyl)₂, —OS(O)₂N(H or C₁-C₂ alkyl)₂, —S(H or C₁-C₂ alkyl), —S(O)C₁-C₂ alkyl, —S(O)₂C₁-C₂ alkyl, —S(O)N(H or C₁-C₂ alkyl)₂, —S(O)₂N(H or C₁-C₂ alkyl)₂, —N(H or C₁-C₂ alkyl)₂, —N(H or C₁-C₂ alkyl)C(O)C₁-C₂ alkyl, —N(H or C₁-C₂ alkyl)C(O)O(H or C₁-C₂ alkyl), —N(H or C₁-C₂ alkyl)C(O)N(H or C₁-C₂ alkyl)₂, —N(H or C₁-C₂ alkyl)S(O)C₁-C₂ alkyl, —N(H or C₁-C₂ alkyl)S(O)₂C₁-C₂ alkyl, —N(H or C₁-C₂ alkyl)S(O)N(H or C₁-C₂ alkyl)₂, —N(H or C₁-C₂ alkyl)S(O)₂N(H or C₁-C₂ alkyl)₂, —C(O)C₁-C₂ alkyl, —C(O)O(H or C₁-C₂ alkyl), —C(O)N(H or C₁-C₂ alkyl)₂, —P(H or C₁-C₂ alkyl)₂, —P(O)(H or C₁-C₂ alkyl)₂, —P(O)₂(H or C₁-C₂ alkyl)₂, —P(O)N(H or C₁-C₂ alkyl)₂, —P(O)₂N(H or C₁-C₂ alkyl)₂, —P(O)O(H or C₁-C₂ alkyl), —P(O)₂O(H or C₁-C₂ alkyl), —CN, or —NO₂;
each R³ and R⁴ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂Rd, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, and R⁵ taken together with the atoms to which they are attached form a C₃-C₆ cycloalkyl or a 4- to 8-membered heterocycloalkyl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁶ and R⁷ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, C₁-C₆ alkyl-C₆-C₁₀ aryl, and 5- to 10-membered heteroaryl; and
n is 2, 3, 4, 5, 6, 7, or 8.

In some embodiments, L does not comprise a —N(R⁵)—. In some embodiments, each L is independently —C(R³)(R⁴)—, —C(O)—, —O—, —S—, —S(O)— or —S(O)₂—, provided that (L)ₙ does not comprise a —O—O—, —S—S—, or —O—S— bond. In some embodiments, R² is not —OR^{a}. In some embodiments, R² is H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, the disclosure provides a compound of the formula II, or a pharmaceutically acceptable salt thereof,

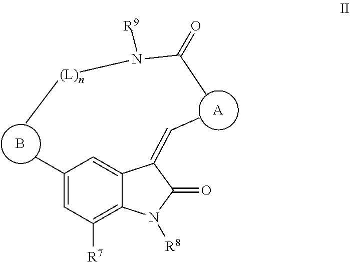

wherein A, B, L, R⁷, R⁸, R⁹, and n are as described herein.

In some embodiments, the disclosure provides a compound of the formula III, or a pharmaceutically acceptable salt thereof,

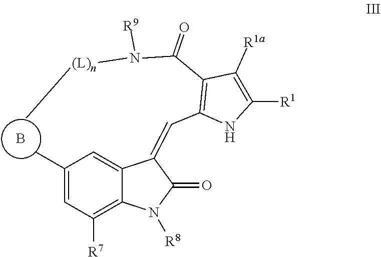

wherein B, L, R¹, R^{1a}, R⁷, R, R⁹, and n are as described herein.

In some embodiments, the disclosure provides a compound of the formula IV, or a pharmaceutically acceptable salt thereof,

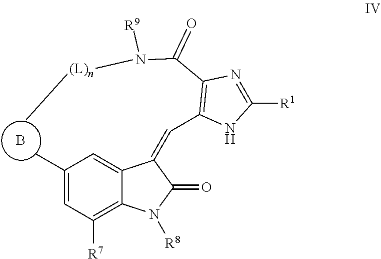

wherein B, L, R¹, R⁷, R⁸, R⁹, and n are as described herein.

In some embodiments, the disclosure provides a compound of the formula V, or a pharmaceutically acceptable salt thereof,

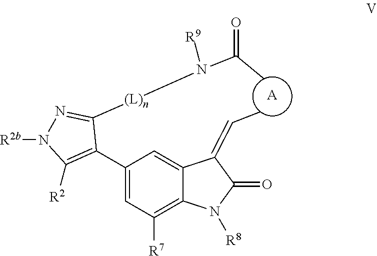

wherein L, R², R^{2b}, R⁷, R⁸, R⁹, and n are as described herein.

In some embodiments, the disclosure provides a compound of the formula VI, or a pharmaceutically acceptable salt thereof,

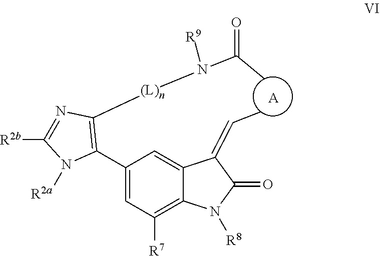

wherein L, R^{2a}, R^{2b}, R⁷, R⁸, R⁹, and n are as described herein.

In some embodiments, the disclosure provides a compound of the formula VII, or a pharmaceutically acceptable salt thereof,

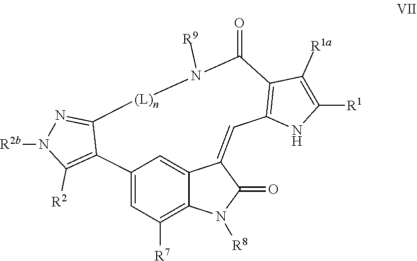

wherein L, R¹, R^{1a}, R², R^{2b}, R⁷, R⁸, R⁹, and n are as described herein.

In some embodiments, the disclosure provides a compound of the formula VIII, or a pharmaceutically acceptable salt thereof,

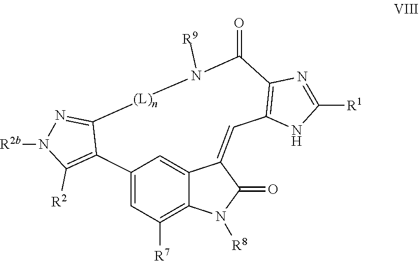

wherein L, R¹, R², R^{2b}, R⁷, R⁸, R⁹, and n are as described herein.

In some embodiments, the disclosure provides a compound of the formula IX, or a pharmaceutically acceptable salt thereof,

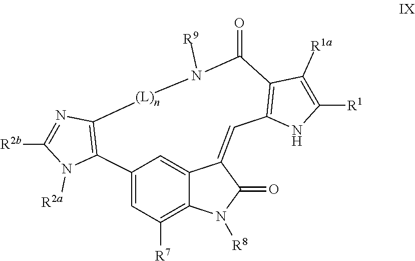

wherein L, R¹, R^{1a}, R^{2a}, R^{2b}, R⁷, R⁸, R⁹, and n are as described herein.

In some embodiments, the disclosure provides a compound of the formula X, or a pharmaceutically acceptable salt thereof,

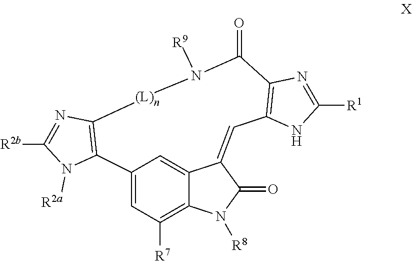

wherein L, R¹, R^{2a}, R^{2b}, R⁷, R⁸, R⁹, and n are as described herein.

In some embodiments, the disclosure provides a compound of the formula XI, or a pharmaceutically acceptable salt thereof,

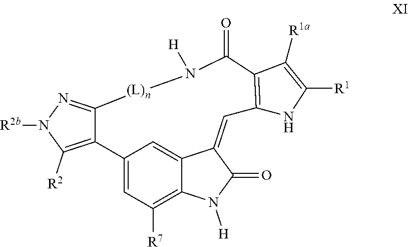

wherein L, R¹, R^{1a}, R², R^{2b}, R⁷, and n are as described herein.

In some embodiments, the disclosure provides a compound of the formula XII, or a pharmaceutically acceptable salt thereof,

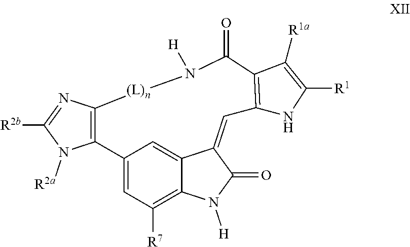

wherein L, R¹, R^{1a}, R^{2a}, R^{2b}, R⁷, and n are as described herein.

In some embodiments, Ring A is

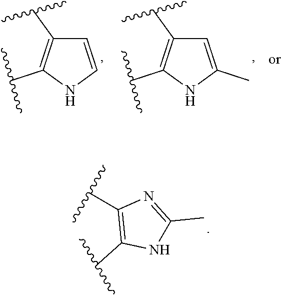

In some embodiments, Ring B is

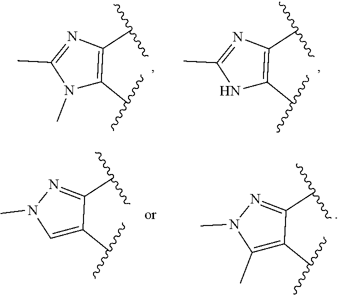

In some embodiments, Ring B is not

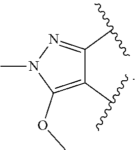 [SMILES: *c1nn(C)c(OC)c1*]

In some embodiments, R⁷ is H, deuterium, C₁-C₆ alkyl, fluoro, chloro, or —CN. In some embodiments, R⁷ is H. In some embodiments, R⁷ is deuterium. In some embodiments, R⁷ is C₁-C₆ alkyl. In some embodiments, R⁷ is —F. In some embodiments, R⁷ —Cl. In some embodiments, R⁷ is —CN.

In some embodiments, Ring B is

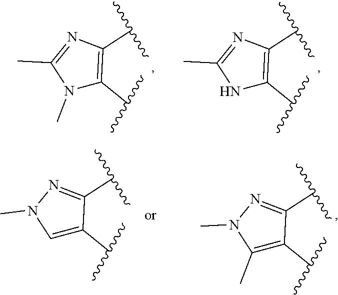

and R⁷ is H. In some embodiments, Ring A is

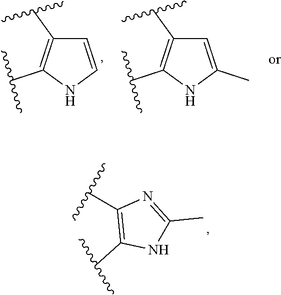

### Ring B is

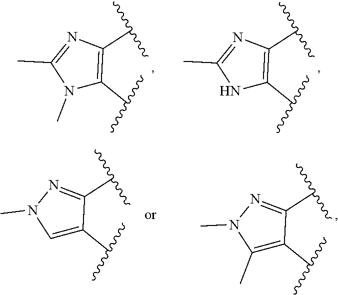

and R⁷ is H. In some embodiments, Ring B is

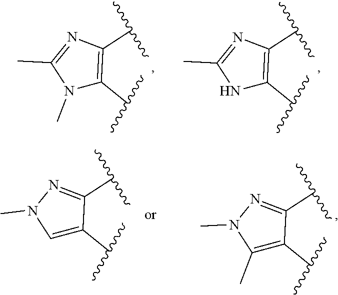

and R⁷ is not H. In some embodiments, Ring A is

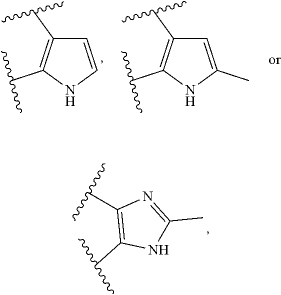

### Ring B is

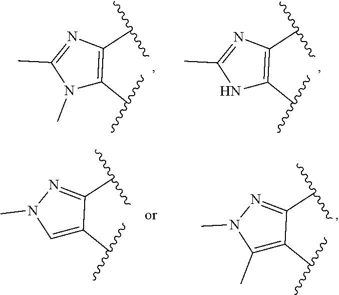

and R⁷ is not H. In some embodiments, Ring B is

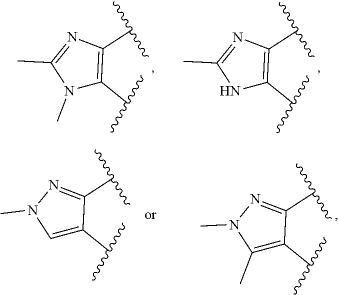

and R⁷ is —F, —Cl or —CN. In some embodiments, Ring B is

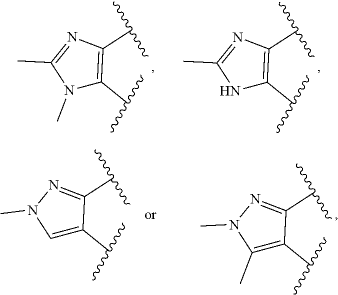

and R⁷ is —Cl or —CN. In some embodiments, Ring A is

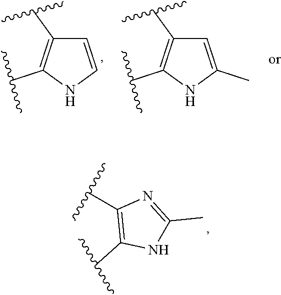

### Ring B is

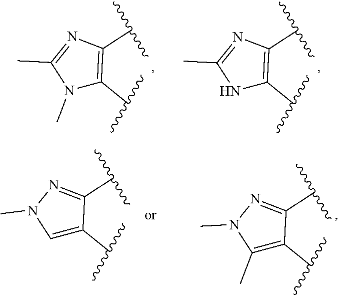

and R⁷ is —F, —Cl or —CN. In some embodiments, Ring A is

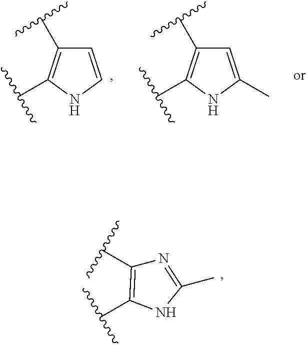

### Ring B is

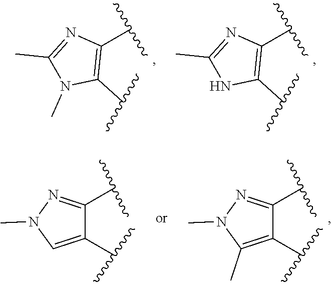

and R⁷ is —Cl or —CN. In some embodiments, Ring B is

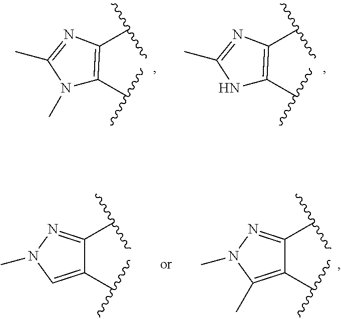

and R⁷ is —F. In some embodiments, Ring B is

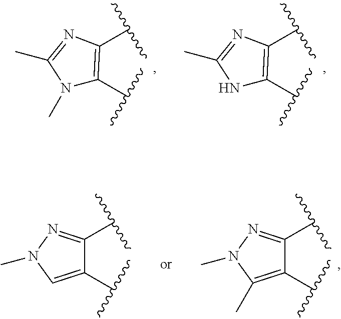

and R⁷ is —Cl. In some embodiments, Ring B is

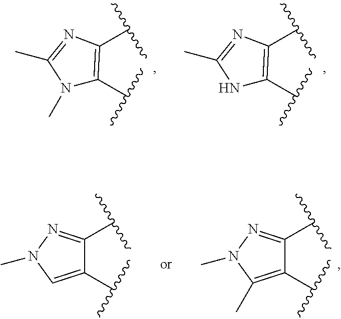

### and R⁷ is —CN.

In some embodiments, Ring A is

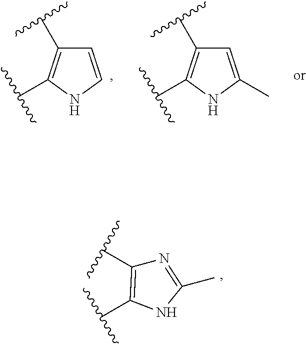

### Ring B is

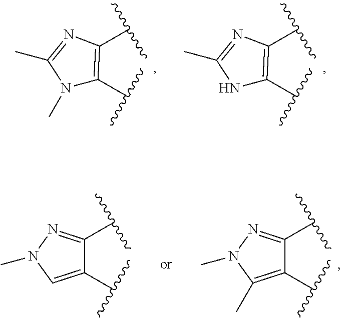

and R⁷ is —F. In some embodiments, Ring A is

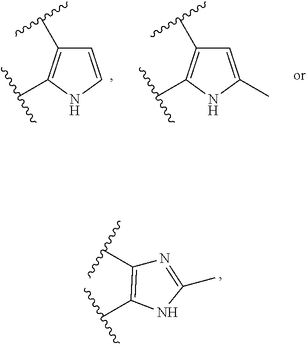

### Ring B is

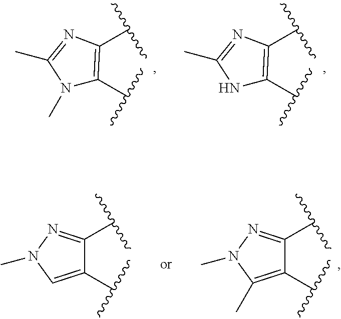

and R⁷ is —Cl. In some embodiments, Ring A is

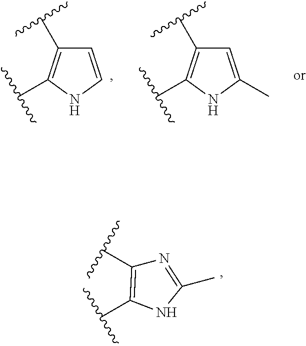

### Ring B is

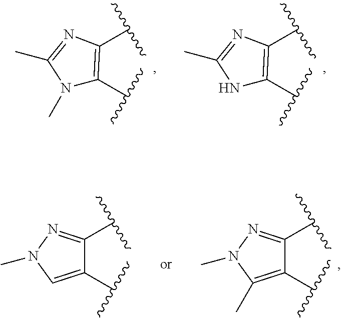

### and R⁷ is —CN.

In certain embodiments of the above aspects, the compound of Formula (I)—(XII) is a compound selected from those species described or exemplified in the detailed description below.

In further aspects, the disclosure relates to a pharmaceutical composition comprising at least one compound of Formula (I)—(XII) or a pharmaceutically acceptable salt thereof. Pharmaceutical compositions according to the disclosure may further comprise a pharmaceutically acceptable excipient.

In further aspects, the disclosure relates to a compound of Formula (I)—(XII), or a pharmaceutically acceptable salt thereof, for use as a medicament.

In further aspects, the disclosure relates to a method of treating disease, such as autoimmune disease comprising administering to a subject in need of such treatment an effective amount of at least one compound of Formula (I)—(XII), or a pharmaceutically acceptable salt thereof.

In further aspects, the disclosure relates to use of a compound of Formula (I)—(XII), or a a pharmaceutically acceptable salt thereof, in the preparation of a medicament for the treatment of disease, such as autoimmune disease, and the use of such compounds and salts for treatment of such diseases.

In further aspects, the disclosure relates to a method of inhibiting a tyrosine kinase, such as TYK2, comprising contacting a cell comprising one or more of kinase with an effective amount of at least one compound of Formula (I)—(XII), or a a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I, or a pharmaceutically acceptable salt thereof,

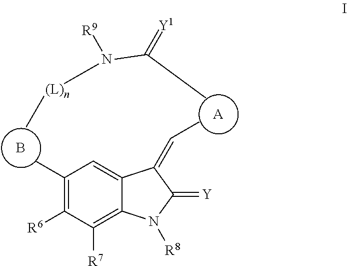

wherein
A is

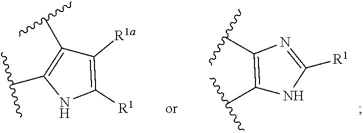

B is

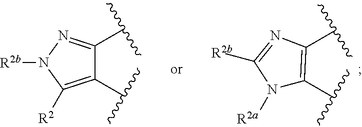

each L is independently —C(R³)(R⁴)—, —C(O)—, —O—, —N(R⁵)—, —S—, —S(O)— or —S(O)₂—, provided that (L)ₙ does not comprise a —O—O—, a —O—S—, a —S—S—, or a —O—N(R⁵)— bond, and (L)ₙ-N(R⁹)— does not comprise a —O—N(R⁹)— a —S—N(R⁹)— bond;
Y and Y¹ are each independently O or S;
each of R¹, R^{1a}, and R² is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R^{2a}, R⁵, R⁸, and R⁹ is independently H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R^{2b} is C₁-C₄ alkyl, C₂-C₄ alkenyl, C₂-C₄ alkynyl, C₃-C₄ cycloalkyl, or 3- to 4-membered heterocycloalkyl, wherein each hydrogen atom in C₁-C₄ alkyl, C₂-C₄ alkenyl, C₂-C₄ alkynyl, C₃-C₄ cycloalkyl, and 3- to 4-membered heterocycloalkyl is independently optionally substituted by deuterium, halogen, —O(H or C₁-C₂ alkyl), —OC(O)C₁-C₂ alkyl, —OC(O)N(H or C₁-C₂ alkyl)₂, —OS(O)C₁-C₂ alkyl, —OS(O)₂C₁-C₂ alkyl, —OS(O)N(H or C₁-C₂ alkyl)₂, —OS(O)₂N(H or C₁-C₂ alkyl)₂, —S(H or C₁-C₂ alkyl), —S(O)C₁-C₂ alkyl, —S(O)₂C₁-C₂ alkyl, —S(O)N(H or C₁-C₂ alkyl)₂, —S(O)₂N(H or C₁-C₂ alkyl)₂, —N(H or C₁-C₂ alkyl)₂, —N(H or C₁-C₂ alkyl)C(O)C₁-C₂ alkyl, —N(H or C₁-C₂ alkyl)C(O)O(H or C₁-C₂ alkyl), —N(H or C₁-C₂ alkyl)C(O)N(H or C₁-C₂ alkyl)₂, —N(H or C₁-C₂ alkyl)S(O)C₁-C₂ alkyl, —N(H or C₁-C₂ alkyl)S(O)₂C₁-C₂ alkyl, —N(H or C₁-C₂ alkyl)S(O)N(H or C₁-C₂ alkyl)₂, —N(H or C₁-C₂ alkyl)S(O)₂N(H or C₁-C₂ alkyl)₂, —C(O)C₁-C₂ alkyl, —C(O)O(H or C₁-C₂ alkyl), —C(O)N(H or C₁-C₂ alkyl)₂, —P(H or C₁-C₂ alkyl)₂, —P(O)(H or C₁-C₂ alkyl)₂, —P(O)₂(H or C₁-C₂ alkyl)₂, —P(O)N(H or C₁-C₂ alkyl)₂, —P(O)₂N(H or C₁-C₂ alkyl)₂, —P(O)O(H or C₁-C₂ alkyl), —P(O)₂O(H or C₁-C₂ alkyl), —CN, or —NO₂;
each R³ and R⁴ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)RC, —S(O)₂R, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂Rd, —NR^{c}S(O)NR^{c}R^{d}, —NR^{c}S(O)₂NR^{c}R^{d}, —C(O)R^{c}, —C(O)ORC, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, and R⁵ taken together with the atoms to which they are attached form a C₃-C₆ cycloalkyl or a 4- to 8-membered heterocycloalkyl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁶ and R⁷ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁶ and R⁷ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 4- to 8-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, C₁-C₆ alkyl-C₆-C₁₀ aryl, and 5- to 10-membered heteroaryl; and
n is 2, 3, 4, 5, 6, 7, or 8.

2. The compound of clause 1 having the formula II

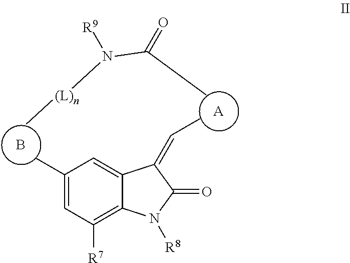

or a pharmaceutically acceptable salt thereof.

3. The compound of clause 1 having the formula III

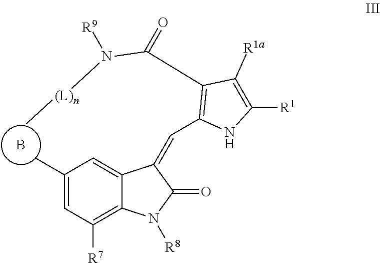

or a pharmaceutically acceptable salt thereof.

4. The compound of clause 1 or 2, having the formula IV

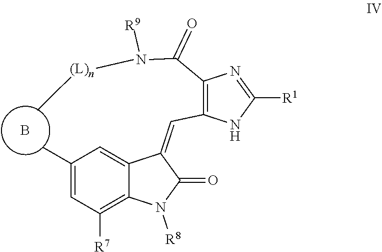

or a pharmaceutically acceptable salt thereof.

5. The compound of clause 1 or 2, having the formula V

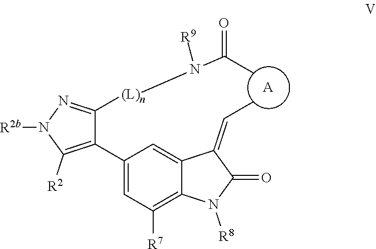

or a pharmaceutically acceptable salt thereof.

6. The compound of clause 1 or 2, having the formula VI

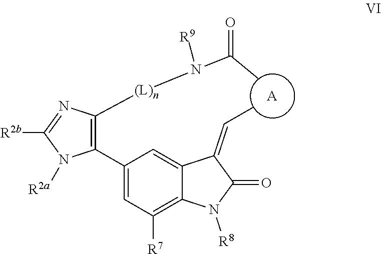

or a pharmaceutically acceptable salt thereof.

7. The compound of any one of clauses 1 to 3, having the formula VII

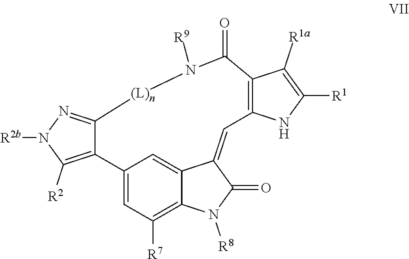

or a pharmaceutically acceptable salt thereof.

8. The compound of clause 1 or 2, having the formula VIII

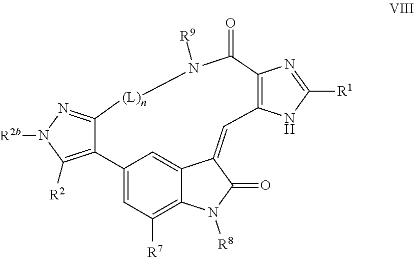

or a pharmaceutically acceptable salt thereof.

9. The compound of any one of clauses 1 to 3, having the formula IX

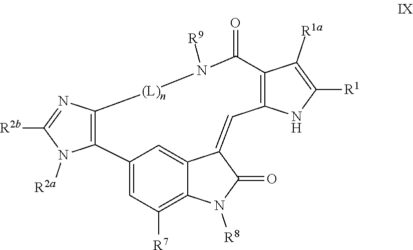

or a pharmaceutically acceptable salt thereof.

10. The compound of clause 1 or 2, having the formula X

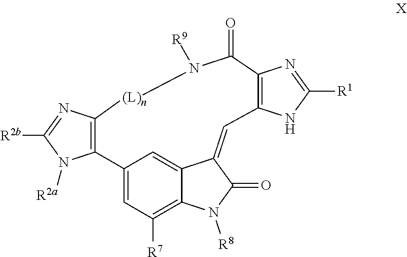

or a pharmaceutically acceptable salt thereof.

11. The compound of any one of clauses 1 to 3, having the formula XI

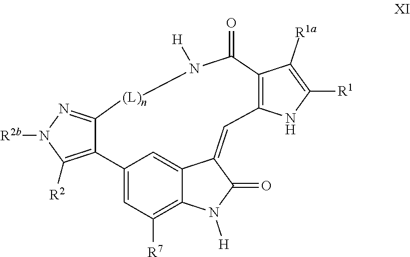

or a pharmaceutically acceptable salt thereof.

12. The compound of any one of clauses 1 to 3, having the formula XII

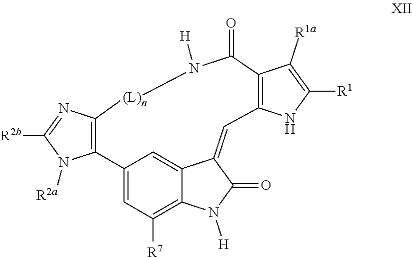

or a pharmaceutically acceptable salt thereof.

13. The compound of any one of the preceding clauses, or a pharmaceutically acceptable salt thereof, wherein R¹, when present, is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or or R¹ is H, —CN or methyl.

14. The compound of any one of clauses 1 to 3, 5 to 7, 9, 11, 12, or 13, or a pharmaceutically acceptable salt thereof, wherein R^{1a}, when present, is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or R^{1a} is H, —CN or methyl.

15. The compound of any one of clauses 1 to 5, 7, 8, 11, 13, or 14, or a pharmaceutically acceptable salt thereof, wherein R², when present, is H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R², when present, is H, —CN, or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R², when present, is H, —CN, or methyl.

16. The compound of any one of the clauses 1 to 4, 6, 9, 10, or 12 to 15, or a pharmaceutically acceptable salt thereof, wherein R^{2a}, when present, is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R^{2a}, when present, is H or methyl.

17. The compound of any one of the preceding clauses, or a pharmaceutically acceptable salt thereof, wherein R^{2b}, when present, is C₁-C₄ alkyl or C₃-C₄ cycloalkyl, wherein each hydrogen atom in C₁-C₆ alkyl and C₃-C₄ cycloalkyl is independently optionally substituted by deuterium or halogen; or R^{2b} is methyl, ethyl, isopropyl, or cyclopropyl.

18. The compound of any one of the preceding clauses, or a pharmaceutically acceptable salt thereof, wherein n is 3.

19. The compound of any one of clauses 1 to 17, or a pharmaceutically acceptable salt thereof, wherein n is 4.

20. The compound of any one of clauses 1 to 17, or a pharmaceutically acceptable salt thereof, wherein n is 5.

21. The compound of any one of clauses 1 to 17, or a pharmaceutically acceptable salt thereof, wherein n is 6.

22. The compound of any one of the preceding clauses, or a pharmaceutically acceptable salt thereof, wherein each L is independently selected from the group consisting of —C(O)—, —O—, —CH₂—, —C(H)(CH₃)—, —C(H)(OH)—, —C(H)(C(O)OR^{c})—, —C(H)(C(O)NR^{c}R^{d})—, —NH—, and —NCH₃—; or each L is independently selected from the group consisting of —C(O)—, —O—, —CH₂—, —C(H)(CH₃)—, —C(H)(OH)—, —C(H)(C(O)OR^{c})—, and —C(H)(C(O)NR^{c}R^{d})—.

23. The compound of any one of clauses 1 or 13 to 22, or a pharmaceutically acceptable salt thereof, wherein Y is O.

24. The compound of any one of clauses 1 or 13 to 23, or a pharmaceutically acceptable salt thereof, wherein Y¹ is O.

25. The compound of any one of clauses 1 or 13 to 25, or a pharmaceutically acceptable salt thereof, wherein R⁶, when present, is independently H, deuterium, fluoro, chloro, —CN, or methyl.

26. The compound of any one of the preceding clauses, or a pharmaceutically acceptable salt thereof, wherein R⁷ is independently H, deuterium, fluoro, chloro, —CN, or methyl.

27. The compound of any one of clauses 1 to 10 or 13 to 26, or a pharmaceutically acceptable salt thereof, wherein R⁸ is H or methyl.

28. The compound of any one of clauses 1 to 10 or 13 to 27, or a pharmaceutically acceptable salt thereof, wherein R⁹ is H or methyl.

29. The compound of any one of the preceding clauses, or a pharmaceutically acceptable salt thereof, wherein -(L)ₙ- is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —CH₂O(CH₂)₃—, —CH₂O(C(CH₃)H)₂—, —(CH₂)₂O(CH₂)₂—, —CH₂OCH₂(C(CH₃)H)—, —(CH₂)₂OCH₂(C(CH₃)H)—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —O—(CH₂)₄—, —O—(CH₂)₂CH(CH₃)— —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N((CH₂)₂CH₃)—(CH₂)₂—, —CH₂N(CH(CH₃)₂)—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—; or —O(CH₂)₂—, —O(CH₂)₃—, —O(CH₂)₄—, —CH₂OCH₂(C(CH₃)H)—, —CH₂O(CH₂)₂—, or —CH₂O(CH₂)₃—; or —O(CH₂)₃—, —CH₂OCH₂(C(CH₃)H)—, or —CH₂O(CH₂)₂—.

30. The compound of clause 1, or a pharmaceutically acceptable salt thereof, selected from the group consisting of [3a(4)Z]-6,9,15,16-tetramethyl-9,10,11,12-tetrahydro-15H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,15,16-tetramethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,10,15-trimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-10,15-dimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10S]-20-chloro-6,9,10,15-tetramethyl-10,11,13,15-tetrahydro-2H-1,17-(ethanediylidene)pyrazolo[4,3-m]dipyrrolo[3,2 f:3′,4′-i][1,4]oxazacyclopentadecine-3,8(5H,9H)-dione;
[3a(4)Z]-20-chloro-6,9,15-trimethyl-10,11,13,15-tetrahydro-2H-1,17-(ethanediylidene)pyrazolo[4,3-m]dipyrrolo[3,2 f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)imidazo[4,5-f]pyrazolo[4,3-m]pyrrolo[3,4-i][1,4]oxazacyclopentadecine-3,8(5H,9H)-dione;
[3a(4)Z,10R]-20-chloro-6,9,10,15-tetramethyl-10,11,13,16-tetrahydro-2H-1,17-(ethanediylidene)imidazo[4,5-m]dipyrrolo[3,2-f:3′,4′-i][1,4]oxazacyclopentadecine-3,8(5H,9H)-dione;
[3a(4)Z,10R]-6,9,10,15-tetramethyl-3,8-dioxo-3,5,8,9,10,11,13,15-octahydro-2H-1,17-(ethanediylidene)pyrazolo[4,3-m]dipyrrolo[3,2 f:3′,4′-i][1,4]oxazacyclopentadecine-20-carbonitrile;
[3a(4)Z,10R]-20-fluoro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione; and
[3a(4)Z,10R]-20-fluoro-6,9,10,15-tetramethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione.

31. A pharmaceutical composition comprising at least one compound of any one of clauses 1 to 30, or a pharmaceutically acceptable salt thereof, and optionally one or more pharmaceutically acceptable excipients.

32. A method of treating disease, such as autoimmune disease, comprising administering to a subject in need of such treatment an effective amount of a compound of any one of clauses 1 to 30, or a pharmaceutically acceptable salt thereof.

33. A compound of any one of clauses 1 to 30, or a pharmaceutically acceptable salt thereof, for use in a method of treating autoimmune disease in a subject.

34. A compound of any one of clauses 1 to 30, or a pharmaceutically acceptable salt thereof, for treating autoimmune disease in a subject.

35. Use of a compound of any one of clauses 1 to 30, or a pharmaceutically acceptable salt thereof, in the manufacture of a medicament for treating autoimmune disease in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2020 (ACD/Labs) or ChemBioDraw Ultra 20.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain mono-valent hydrocarbon group. The term “alkylene” refers to a straight- or branched-chain di-valent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. The term “alkenylene” refers to a straight- or branched-chain di-valent hydrocarbon group having one or more double bonds. In some embodiments, it can be advantageous to limit the number of atoms in an “alkenyl” or “alkenylene” to a specific range of atoms, such as C₂-C₂₀ alkenyl or C₂-C₂₀ alkenylene, C₂-C₁₂ alkenyl or C₂-C₁₂ alkenylene, or C₂-C₆ alkenyl or C₂-C₆ alkenylene. Examples of alkenyl groups include ethenyl (or vinyl), allyl, and but-3-en-1-yl. Examples of alkenylene groups include ethenylene (or vinylene) (—CH═CH—), n-propenylene (—CH═CHCH₂—), iso-propenylene (—CH═CH(CH₃)—), and and the like. Included within this term are cis and trans isomers and mixtures thereof. It will be appreciated that an alkenyl or alkenylene group can be unsubstituted or substituted as described herein. An alkenyl or alkenylene group can be substituted with any of the substituents in the various embodiments described herein, including one or more of such substituents.

The term “alkynyl” refers to a straight- or branched-chain mono-valent hydrocarbon group having one or more triple bonds. The term “alkynylene” refers to a straight- or branched-chain di-valent hydrocarbon group having one or more triple bonds. In some embodiments, it can be advantageous to limit the number of atoms in an “alkynyl” or “alkynylene” to a specific range of atoms, such as C₂-C₂₀ alkynyl or C₂-C₂₀ alkynylene, C₂-C₁₂ alkynyl or C₂-C₁₂ alkynylene, or C₂-C₆ alkynyl or C₂-C₆ alkynylene. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl or alkynylene group can be unsubstituted or substituted as described herein. An alkynyl or alkynylene group can be substituted with any of the substituents in the various embodiments described herein, including one or more of such substituents.

The term “cycloalkyl” refers to a saturated or partially saturated, monocyclic or polycyclic mono-valent carbocycle. The term “cycloalkylene” refers to a saturated or partially saturated, monocyclic or polycyclic di-valent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include mono-valent radicals of the following entities, while cycloalkylene groups include di-valent radicals of the following entities, in the form of properly bonded moieties:

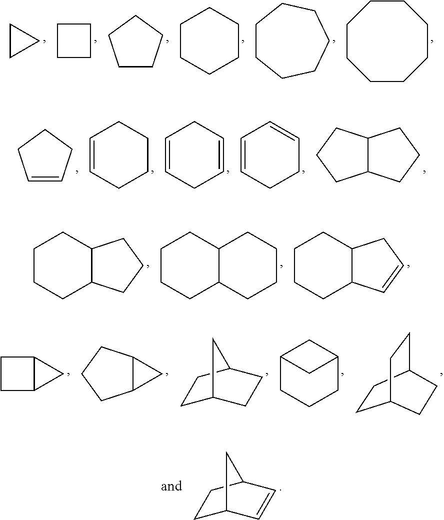

In particular, a cyclopropyl moiety can be depicted by the structural formula

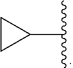 [SMILES: *C1CC1]

In particular, a cyclopropylene moiety can be depicted by the structural formula

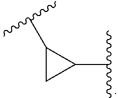 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F. The term “haloalkylene” refers to an alkyl group with one or more halo substituents. Examples of haloalkyl groups include —CF₂—, —C(H)(F)—, —C(H)(Br)—, —CH₂CF₂—, and —CH₂C(H)(F)—.

The term “aryl” refers to a mono-valent all-carbon monocyclic or fused-ring polycyclic group having a completely conjugated pi-electron system. The term “arylene” refers to a mono-valent all-carbon monocyclic or fused-ring polycyclic group having a completely conjugated pi-electron system. In some embodiments, it can be advantageous to limit the number of atoms in an “aryl” or “arylene” to a specific range of atoms, such as mono-valent all-carbon monocyclic or fused-ring polycyclic groups of 6 to 14 carbon atoms (C₆-C₁₄ aryl), mono-valent all-carbon monocyclic or fused-ring polycyclic groups of 6 to 10 carbon atoms (C₆-C₁₀ aryl), di-valent all-carbon monocyclic or fused-ring polycyclic groups of 6 to 14 carbon atoms (C₆-C₁₄ arylene), di-valent all-carbon monocyclic or fused-ring polycyclic groups of 6 to 10 carbon atoms (C₆-C₁₀ arylene). Examples, without limitation, of aryl groups are phenyl, naphthalenyl and anthracenyl. Examples, without limitation, of aryl groups are phenylene, naphthalenylene and anthracenylene. It will be appreciated that an aryl or arylene group can be unsubstituted or substituted as described herein. An aryl or arylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a mono-valent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 8 ring atoms (4- to 8-membered), or 5 to 7 ring atoms (5- to 7-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group on a carbon ring member or up to two oxo groups on sulfur ring members. Illustrative examples of heterocycloalkyl groups include mono-valent radicals of the following entities, while heterocycloalkylene groups include di-valent radicals of the following entities, in the form of properly bonded moieties:

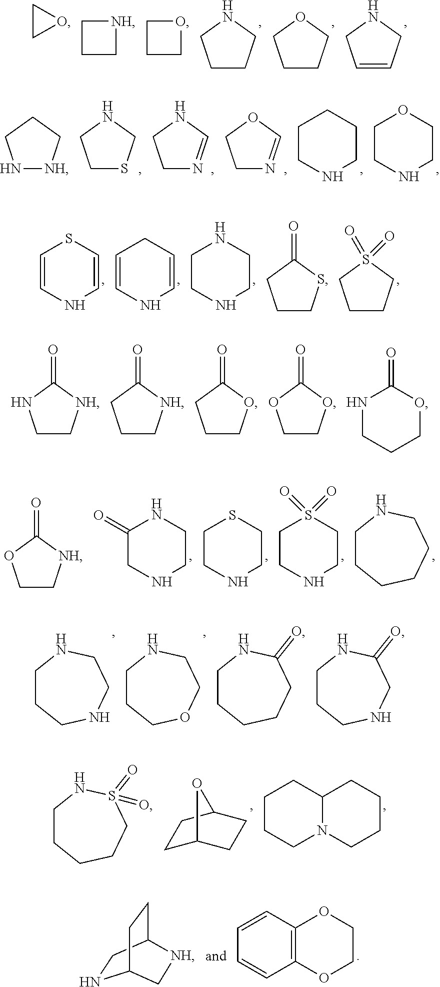

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2,5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a di-valent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5- to 10-ring atoms wherein at least one ring atom is a heteroatom, such as N, O, or S. Illustrative examples of 5- to 10-membered heteroaryl groups include mono-valent radicals of the following entities, while examples of 5- to 10-membered heteroarylene groups include di-valent radicals of the following entities, in the form of properly bonded moieties:

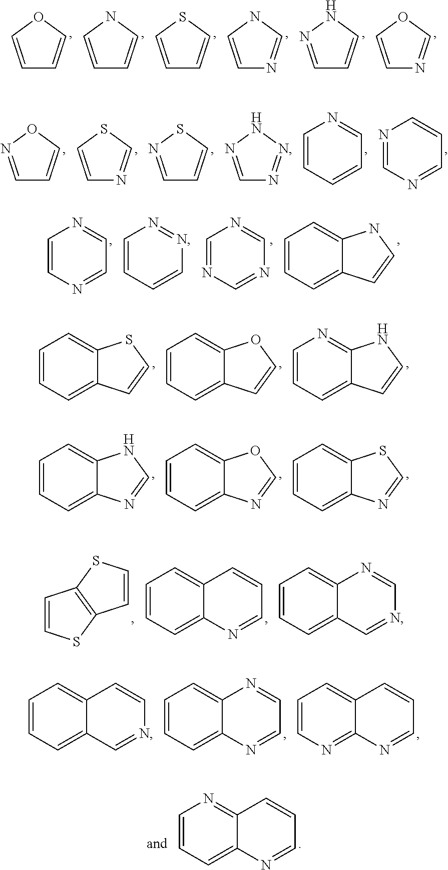

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. Non-limiting examples of six-membered heteroarylene groups include divalent radicals of pyridine, pyrazine, pyrimidine, pyridazine, or triazine. A “bicyclic heteroaryl” or “bicyclic heteroarylene” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, indole, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole. Non-limiting examples of bicyclic heteroarylene groups include divalent radicals of quinoline, isoquinoline, quinazoline, quinoxaline, indole, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole.

In particular, a pyrrolyl moiety can be depicted by the structural formula

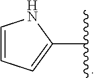 [SMILES: *c1ccc[nH]1]

In particular, a pyrrolylene moiety can be depicted by the structural formula

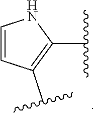 [SMILES: *c1cc[nH]c1*]

In particular, a pyrazolyl moiety can be depicted by the structural formula

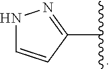 [SMILES: *c1cc[nH]n1]

In particular, a pyrazolylene moiety can be depicted by the structural formula

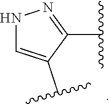 [SMILES: *c1c[nH]nc1*]

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

Certain chemical entities of Formula (I)—(XII) may be depicted in two or more tautomeric forms. Any and all alternative tautomers are included within the scope of these formulas, and no inference should be made as to whether the chemical entity exists as the tautomeric form in which it is drawn. It will be understood that the chemical entities described herein, and their constituent rings A, B, etc. can exist in different tautomeric forms. It will be readily appreciated by one of skill in the art that because of rapid interconversion, tautomers can generally be considered to be the same chemical compound. Examples of tautomers include but are not limited to enol-keto tautomers, amine-imine tutomers, and the like.

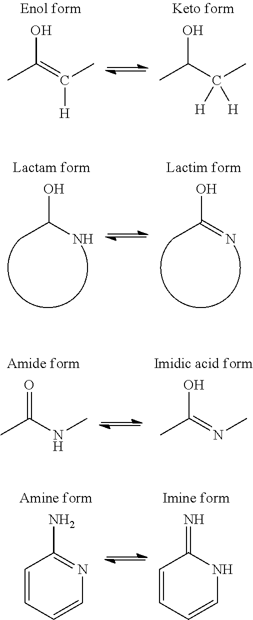

In particular, a ring option of indolin-2-oneylene can exist as the following tautomers

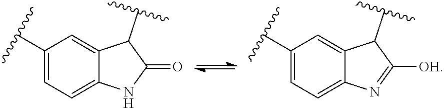 [SMILES: *c1ccc2c(c1)C(*)C(=O)N2 ; *c1ccc2c(c1)C(*)C(O)=N2]

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ₋ⱼ” with j>i, when applied herein to a class of substituents, is meant to refer to embodiments of this disclosure for which each and every one of the number of atom members, from i to j including i and j, is independently realized. By way of example, the term C₁₋₃ refers independently to embodiments that have one carbon member (C₁), embodiments that have two carbon members (C₂), and embodiments that have three carbon members (C₃).

Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member. For example, in certain embodiments, where applicable, a compound portion -(L)ₙ- having the formula —CH₂OCH₂(C(CH₃)H)—, connecting two groups, A and B, will be understood that —CH₂OCH₂(C(CH₃)H)—, can include both of the embodiments A-CH₂OCH₂(C(CH₃)H)—B and B—CH₂OCH₂(C(CH₃)H)-A. More particularly in the present case, compounds of the formula (I)—(XII) having a compound portion -(L)ₙ- of the formula —CH₂OCH₂(C(CH₃)H)— connecting groups —B— and —NR⁹— will be understood to include both embodiments B—CH₂OCH₂(C(CH₃)H)—NR⁹ and —NR⁹—CH₂OCH₂(C(CH₃)H)—B.

The disclosure also includes pharmaceutically acceptable salts of the compounds represented by Formula (I)—(XII), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)—(XII) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)—(XII), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)—(XII)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (I)—(XII), and uses of such metabolites in the methods of the disclosure. A “pharmaceutically active metabolite” means a pharmacologically active product of metabolism in the body of a compound of Formula (I)—(XII) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “protecting group” or “PG” refers to any group as commonly known to one of ordinary skill in the art that can be introduced into a molecule by chemical modification of a functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N.J: Wiley-Interscience. One of skill in the art will readily appreciate the chemical process conditions under which such protecting groups can be installed on a functional group. Suitable amine protecting groups useful in connection with the present disclosure include, but are not limited to, 9-Fluorenylmethyl-carbonyl (FMOC), t-butylcarbonyl (Boc), benzyloxycarbonyl (Cbz), acetyl (Ac), trifluoroacetyl, phthalimide, benzyl (Bn), triphenylmethyl (trityl, Tr), benzylidene, and p-toluenesulfonyl (tosylamide, Ts).

### REPRESENTATIVE EMBODIMENTS

In some embodiments, the disclosure provides a compound of the formula I, or a pharmaceutically acceptable salt thereof,

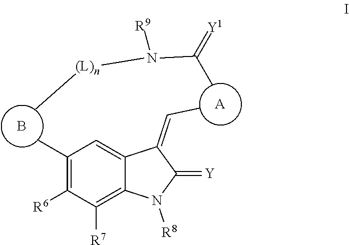

wherein A, B, L, R⁶, R⁷, R⁸, R⁹, Y, Y¹, and n are as described herein.

In some embodiments, the disclosure provides a compound of the formula II, or a pharmaceutically acceptable salt thereof,

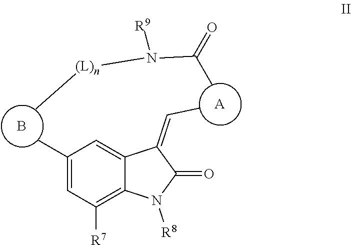

wherein A, B, L, R⁷, R⁸, R⁹, and n are as described herein.

In some embodiments, the disclosure provides a compound of the formula III, or a pharmaceutically acceptable salt thereof,

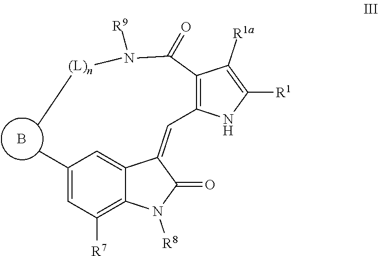

wherein B, L, R¹, R^{1a}, R⁷, R⁸, R⁹, and n are as described herein.

In some embodiments, the disclosure provides a compound of the formula IV, or a pharmaceutically acceptable salt thereof,

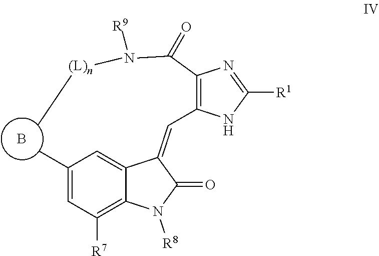

wherein B, L, R^{1a}, R⁷, R⁸, R⁹, and n are as described herein.

In some embodiments, the disclosure provides a compound of the formula V, or a pharmaceutically acceptable salt thereof,

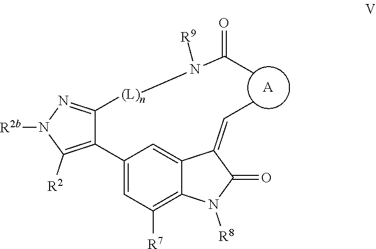

wherein A, L, R², R^{2b}, R⁷, R⁸, R⁹, and n are as described herein.

In some embodiments, the disclosure provides a compound of the formula VI, or a pharmaceutically acceptable salt thereof,

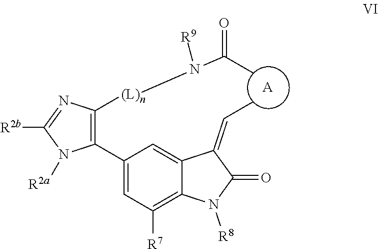

wherein A, L, R^{2a}, R^{2b}, R⁷, R⁸, R⁹, and n are as described herein.

In some embodiments, the disclosure provides a compound of the formula VII, or a pharmaceutically acceptable salt thereof,

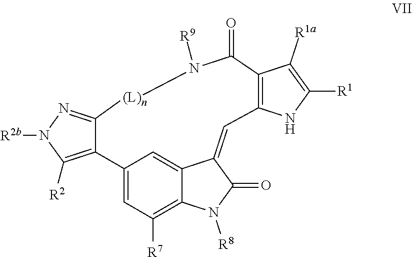

wherein L, R¹, R^{1a}, R², R^{2b}, R⁷, R⁸, R⁹, and n are as described herein.

In some embodiments, the disclosure provides a compound of the formula VIII, or a pharmaceutically acceptable salt thereof,

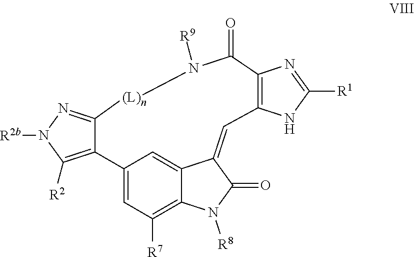

wherein L, R¹, R², R^{2b}, R⁷, R⁸, R⁹, and n are as described herein.

In some embodiments, the disclosure provides a compound of the formula IX, or a pharmaceutically acceptable salt thereof,

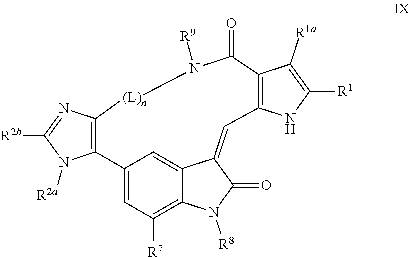

wherein L, R¹, R^{1a}, R^{2a}, R^{2b}, R⁷, R⁸, R⁹, and n are as described herein.

In some embodiments, the disclosure provides a compound of the formula X, or a pharmaceutically acceptable salt thereof,

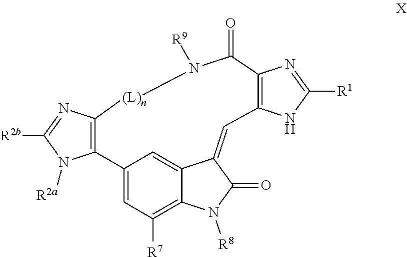

wherein L, R¹, R^{2a}, R^{2b}, R⁷, R⁸, R⁹, and n are as described herein.

In some embodiments, the disclosure provides a compound of the formula XI, or a pharmaceutically acceptable salt thereof,

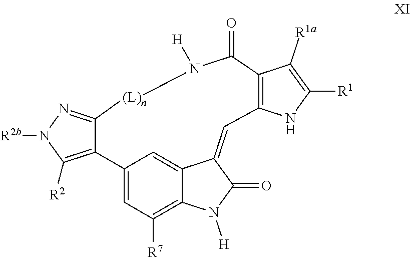

wherein L, R¹, R^{1a}, R², R^{2b}, R⁷, and n are as described herein.

In some embodiments, the disclosure provides a compound of the formula XII, or a pharmaceutically acceptable salt thereof,

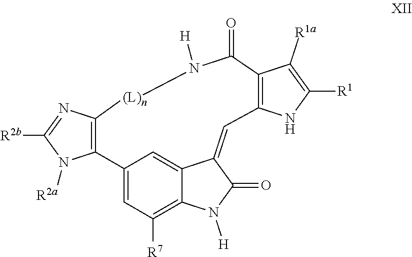

wherein L, R¹, R^{1a}, R^{2a}, R^{2b}, R⁷, and n are as described herein.

In some embodiments, Ring A is

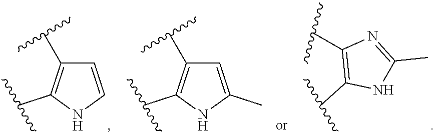

In some embodiments, Ring B is

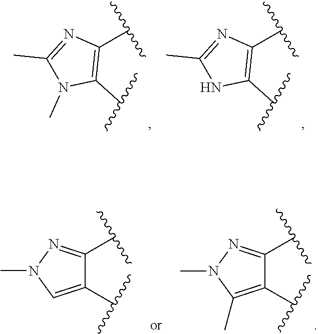

In some embodiments, Ring B is

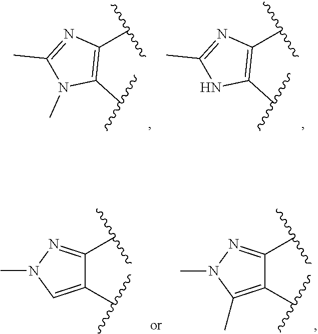

and R⁷ is H. In some embodiments, Ring A is

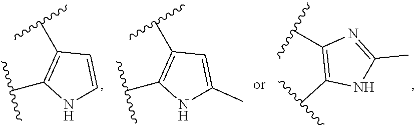

### Ring B is

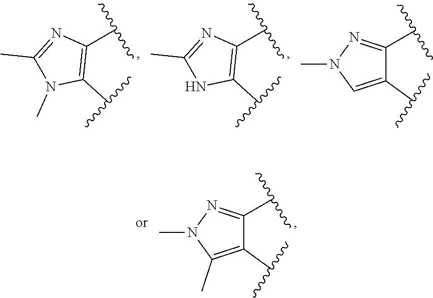

and R⁷ is H. In some embodiments, Ring B is

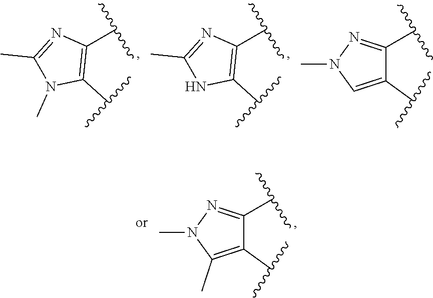

and R⁷ is not H. In some embodiments, Ring A is

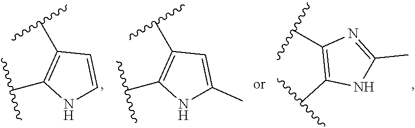

### Ring B is

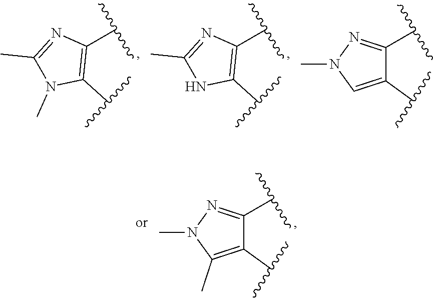

and R⁷ is not H. In some embodiments, Ring B is

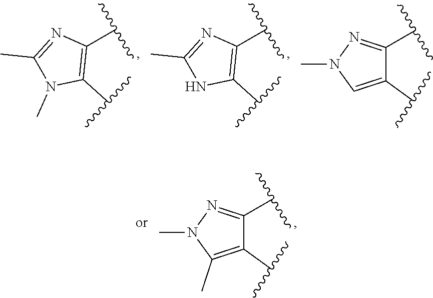

and R⁷ is —Cl or —CN. In some embodiments, Ring A is

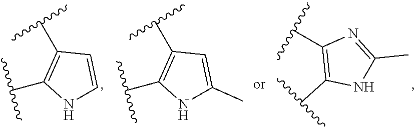

### Ring B is

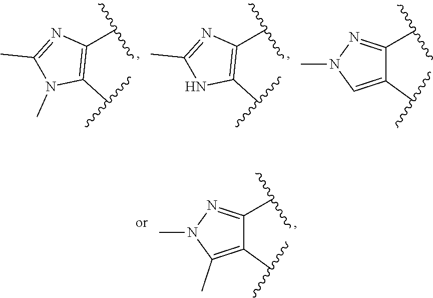

and R⁷ is —Cl or —CN. In some embodiments, Ring B is

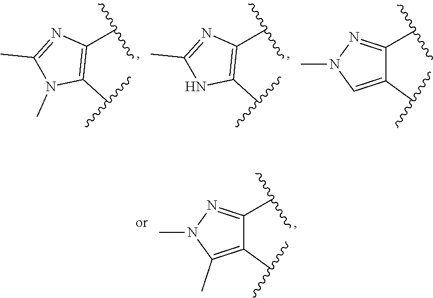

and R⁷ is —Cl. In some embodiments, Ring A is

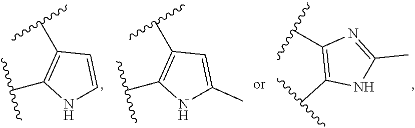

### Ring B is

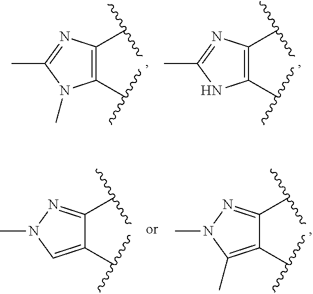

and R⁷ is —Cl. In some embodiments, Ring B is

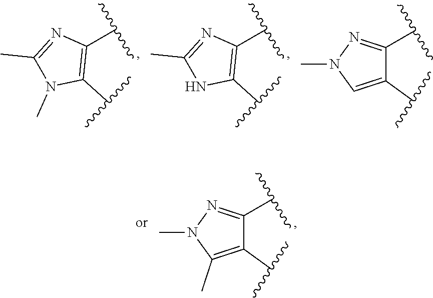

and R⁷ is —CN. In some embodiments, Ring A is

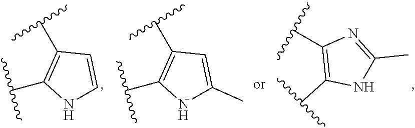

### Ring B is

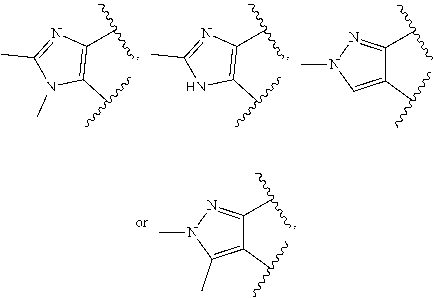

### and R⁷ is —CN.

In some embodiments, R¹ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂

In some embodiments, R¹ is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R¹ is H, —CN, or methyl.

In some embodiments, R^{1a} is H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R^{1a} is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R^{1a}, when present, is H, —CN, or methyl.

In some embodiments, R² is H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R² is independently H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R² is H, —CN, or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R² is H, —CN, or methyl.

In some embodiments, R^{2a} is H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R^{2a} is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R^{2a} is H or methyl.

In some embodiments, R^{2b} is C₁-C₄ alkyl, C₂-C₄ alkenyl, C₂-C₄ alkynyl, C₃-C₄ cycloalkyl, or 3- to 4-membered heterocycloalkyl, wherein each hydrogen atom in C₁-C₄ alkyl, C₂-C₄ alkenyl, C₂-C₄ alkynyl, C₃-C₄ cycloalkyl, and 3- to 4-membered heterocycloalkyl is independently optionally substituted by deuterium, halogen, —O(H or C₁-C₂ alkyl), —OC(O)C₁-C₂ alkyl, —OC(O)N(H or C₁-C₂ alkyl)₂, —OS(O)C₁-C₂ alkyl, —OS(O)₂C₁-C₂ alkyl, —OS(O)N(H or C₁-C₂ alkyl)₂, —OS(O)₂N(H or C₁-C₂ alkyl)₂, —S(H or C₁-C₂ alkyl), —S(O)C₁-C₂ alkyl, —S(O)₂C₁-C₂ alkyl, —S(O)N(H or C₁-C₂ alkyl)₂, —S(O)₂N(H or C₁-C₂ alkyl)₂, —N(H or C₁-C₂ alkyl)₂, —N(H or C₁-C₂ alkyl)C(O)C₁-C₂ alkyl, —N(H or C₁-C₂ alkyl)C(O)O(H or C₁-C₂ alkyl), —N(H or C₁-C₂ alkyl)C(O)N(H or C₁-C₂ alkyl)₂, —N(H or C₁-C₂ alkyl)S(O)C₁-C₂ alkyl, —N(H or C₁-C₂ alkyl)S(O)₂C₁-C₂ alkyl, —N(H or C₁-C₂ alkyl)S(O)N(H or C₁-C₂ alkyl)₂, —N(H or C₁-C₂ alkyl)S(O)₂N(H or C₁-C₂ alkyl)₂, —C(O)C₁-C₂ alkyl, —C(O)O(H or C₁-C₂ alkyl), —C(O)N(H or C₁-C₂ alkyl)₂, —P(H or C₁-C₂ alkyl)₂, —P(O)(H or C₁-C₂ alkyl)₂, —P(O)₂(H or C₁-C₂ alkyl)₂, —P(O)N(H or C₁-C₂ alkyl)₂, —P(O)₂N(H or C₁-C₂ alkyl)₂, —P(O)O(H or C₁-C₂ alkyl), —P(O)₂O(H or C₁-C₂ alkyl), —CN, or —NO₂.

In some embodiments, R^{2b} is C₁-C₄ alkyl or C₃-C₄ cycloalkyl, wherein each hydrogen atom in C₁-C₆ alkyl and C₃-C₄ cycloalkyl is independently optionally substituted by deuterium or halogen. In some embodiments, R^{2b} is methyl, ethyl, isopropyl, or cyclopropyl.

In some embodiments, each L is independently —C(R³)(R⁴)—, —C(O)—, —O—, —N(R⁵)—, —S—, —S(O)— or —S(O)₂—, provided that (L)ₙ does not comprise a —O—O—, a —O—S—, a a —S—S—, or a —O—N(R⁵)— bond. In some embodiments, each L is independently —C(R³)(R⁴)—, —C(O)—, —O—, —S—, —S(O)— or —S(O)₂—, provided that (L)ₙ does not comprise a —O—O—, —S—S—, or —O—S— bond. In some embodiments, L does not comprise a —N(R⁵)—.

In some embodiments, each R³, and R⁴ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)RC, —S(O)₂RC, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂Rd, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, and R⁵ taken together with the atoms to which they are attached form a C₃-C₆ cycloalkyl or a 4- to 8-membered heterocycloalkyl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each L is independently selected from the group consisting of —C(O)—, —O—, —CH₂—, —C(H)(CH₃)—, —C(H)(OH)—, —NH—, and —NCH₃—. In some embodiments, -(L)ₙ- is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —CH₂O(CH₂)₃—, —CH₂O(C(CH₃)H)₂—, —(CH₂)₂O(CH₂)₂—, —CH₂OCH₂(C(CH₃)H)—, —(CH₂)₂OCH₂(C(CH₃)H)—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —O—(CH₂)₄—, —O—(CH₂)₂CH(CH₃)— —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N((CH₂)₂CH₃)—(CH₂)₂—, —CH₂N(CH(CH₃)₂)—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—. In some embodiments, —Z-(L)ₙ-Z¹— does not comprise an —O—O—, a —O—S—, or an —O—N(R^{x})— bond. In some embodiments, -(L)ₙ- is —O(CH₂)₂—, —O—(CH₂)₃—, —O—(CH₂)₄—, —CH₂OCH₂(C(CH₃)(H))—, —CH₂O(CH₂)₂—, or —CH₂O(CH₂)₃—. In some embodiments, -(L)ₙ- is —O(CH₂)₃—, —CH₂OCH₂(C(CH₃)(H))—, or —CH₂O(CH₂)₂—.

In some embodiments, R^{1a}, R^{2a}, R⁵, R⁸, and R⁹ are each independently H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R^{1a} is H or methyl. In some embodiments, R^{2a} is H or methyl. In some embodiments, R⁵ is H or methyl. In some embodiments, R⁸ is H or methyl. In some embodiments, R⁹ is H or methyl.

In some embodiments, each of R⁶ and R⁷ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁶ and R⁷ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 4- to 8-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R⁶ is H, deuterium, fluoro, chloro, —CN, or C₁-C₆ alkyl, such as methyl or ethyl. In some embodiments, R⁷ is H, deuterium, fluoro, chloro, —CN, or C₁-C₆ alkyl, such as methyl or ethyl. In some embodiments, R⁶ is H or deuterium, and R⁷ is deuterium, fluoro, chloro, —CN, or C₁-C₆ alkyl, such as methyl or ethyl. In some embodiments, R⁷ is H. In some embodiments, R⁷ is deuterium. In some embodiments, R⁷ is C₁-C₆ alkyl, such as methyl or ethyl. In some embodiments, R⁷ is —F. In some embodiments, R⁷ —Cl. In some embodiments, R⁷ is —CN.

In some embodiments, n is 2, 3, 4, 5, 6, 7, or 8. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, the disclosure provides a compound selected from the group consisting of [3a(4)Z]-6,9,15,16-tetramethyl-9,10,11,12-tetrahydro-15H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,15,16-tetramethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,10,15-trimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-10,15-dimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)pyrazolo[4,3-m]dipyrrolo[3,2 f:3′,4′-i][1,4]oxazacyclopentadecine-3,8(5H,9H)-dione;
[3a(4)Z,10S]-20-chloro-6,9,10,15-tetramethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-20-chloro-6,9,15-trimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)imidazo[4,5-f]pyrazolo[4,3-m]pyrrolo[3,4-i][1,4]oxazacyclopentadecine-3,8(5H,9H)-dione;
[3a(4)Z,10R]-20-chloro-6,9,10,15-tetramethyl-10,11,13,16-tetrahydro-2H-1,17-(ethanediylidene)imidazo[4,5-m]dipyrrolo[3,2-f:3′,4′-i][1,4]oxazacyclopentadecine-3,8(5H,9H)-dione;
[3a(4)Z,10R]-6,9,10,15-tetramethyl-3,8-dioxo-3,5,8,9,10,11,13,15-octahydro-2H-1,17-(ethanediylidene)pyrazolo[4,3-m]dipyrrolo[3,2-f:3′,4′-i][1,4]oxazacyclopentadecine-20-carbonitrile;
[3a(4)Z,10R]-20-fluoro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione; and
[3a(4)Z,10R]-20-fluoro-6,9,10,15-tetramethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione;
   or a pharmaceutically acceptable salt thereof.

The following represent illustrative embodiments of compounds of Formula (I):

| Cpd. # | Structure | ACD Name |
|---|---|---|
| 1 | 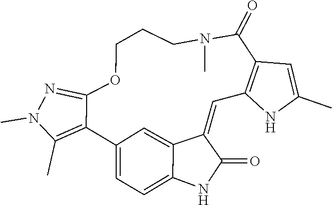 | [3a(4)Z]-6,9,15,16-tetramethyl- 9,10,11,12-tetrahydro-15H-1,17- (ethanediylidene)pyrazolo[4,3- n]dipyrrolo[3,2-g:3′,4′- j][1,5]oxazacyclopentadecine- 3,8(2H,5H)-dione |
| 2 | 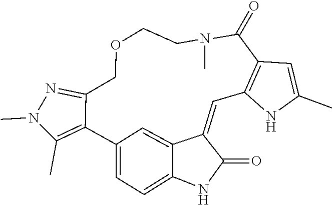 | [3a(4)Z]-6,9,15,16-tetramethyl- 10,11,13,15-tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 3 | 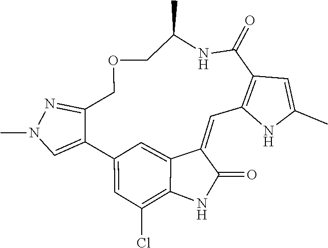 | [3a(4)Z,10R]-20-chloro-6,10,15- trimethyl-10,11,13,15-tetrahydro-2H- 1,17-(ethanediylidene)pyrazolo[4,3- m]dipyrrolo[3,2-f:3′,4′- i][1,4]oxazacyclopentadecine- 3,8(5H,9H)-dione |
| 4 | 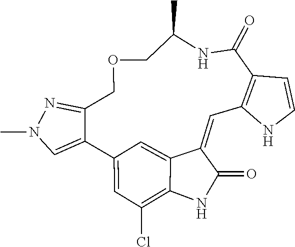 [SMILES: C[C@@H]1COCc2nn(C)cc2-c2cc(Cl)c3c(c2)/C(=C/c2[nH]ccc2C(=O)N1)C(=O)N3] | [3a(4)Z,10R]-20-chloro-10,15-dimethyl- 10,11,13,15-tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 5 | 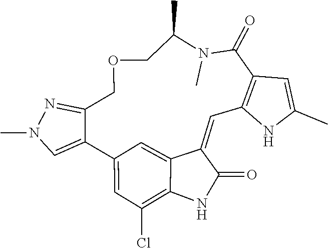 | [3a(4)Z,10R]-20-chloro-6,9,10,15- tetramethyl-10,11,13,15-tetrahydro-2H- 1,17-(ethanediylidene)pyrazolo[4,3- m]dipyrrolo[3,2-f:3′,4′- i][1,4]oxazacyclopentadecine- 3,8(5H,9H)-dione |
| 6 | 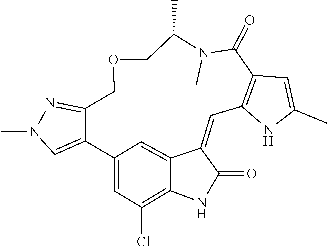 | [3a(4)Z,10S]-20-chloro-6,9,10,15- tetramethyl-10,11,13,15-tetrahydro-2H- 1,17-(ethanediylidene)pyrazolo[4,3- m]dipyrrolo[3,2-f:3′,4′- i][1,4]oxazacyclopentadecine- 3,8(5H,9H)-dione |
| 7 | 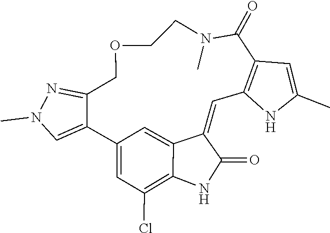 | [3a(4)Z]-20-chloro-6,9,15-trimethyl- 10,11,13,15-tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 8 | 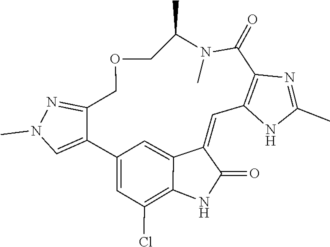 | [3a(4)Z,10R]-20-chloro-6,9,10,15- tetramethyl-10,11,13,15-tetrahydro-2H- 1,17-(ethanediylidene)imidazo[4,5- f]pyrazolo[4,3-m]pyrrolo[3,4- i][1,4]oxazacyclopentadecine- 3,8(5H,9H)-dione |
| 9 | 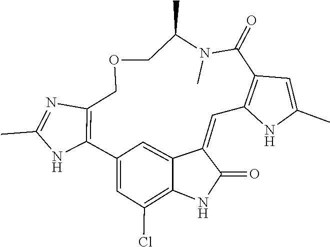 | [3a(4)Z,10R]-20-chloro-6,9,10,15- tetramethyl-10,11,13,16-tetrahydro-2H- 1,17-(ethanediylidene)imidazo[4,5- m]dipyrrolo[3,2-f:3′,4′- i][1,4]oxazacyclopentadecine- 3,8(5H,9H)-dione |
| 10 | 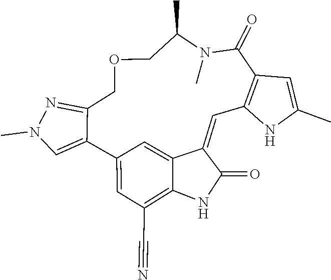 | [3a(4)Z,10R]-6,9,10,15-tetramethyl-3,8- dioxo-3,5,8,9,10,11,13,15-octahydro-2H- 1,17-(ethanediylidene)pyrazolo[4,3- m]dipyrrolo[3,2-f:3′,4′- i][1,4]oxazacyclopentadecine-20- carbonitrile |
| 11 | 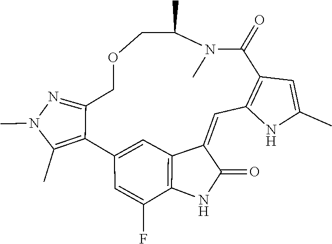 | [3a(4)Z,10R]-20-fluoro-6,9,10,15,16- pentamethyl-10,11,13,15-tetrahydro-2H- 1,17-ethenopyrazolo[4,3- m]dipyrrolo[3,2-f:3′,4′- i][1,4]oxazacyclopentadecine- 3,8(5H,9H)-dione |
| 12 | 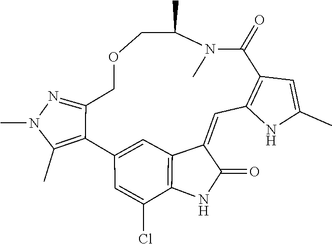 | [3a(4)Z,10R]-20-chloro-6,9,10,15,16- pentamethyl-10,11,13,15-tetrahydro-2H- 1,17-ethenopyrazolo[4,3- m]dipyrrolo[3,2-f:3′,4′- i][1,4]oxazacyclopentadecine- 3,8(5H,9H)-dione |
| 13 | 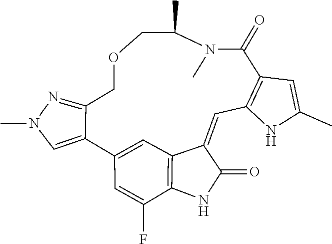 | [3a(4)Z,10R]-20-fluoro-6,9,10,15- tetramethyl-10,11,13,15-tetrahydro-2H- 1,17-ethenopyrazolo[4,3- m]dipyrrolo[3,2-f:3′,4′- i][1,4]oxazacyclopentadecine- 3,8(5H,9H)-dione |

and pharmaceutically acceptable salts thereof.

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include autoimmune diseases and inflammation. Autoimmune diseases include, for example, rheumatoid arthritis, psoriasis, inflammatory bowel disease and systemic lupus erythematosus, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target TYK2. Thus, these compounds and pharmaceutical compositions can be used to prevent, reverse, slow, or inhibit the activity of TYK2. In preferred embodiments, methods of treatment target autoimmune disease. In other embodiments, methods are for treating autoimmune disease, such as rheumatoid arthritis, psoriasis, inflammatory bowel disease and systemic lupus erythematosus, Sjogren syndrome, Type I diabetes, and lupus.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a autoimmune disease cell with abnormal signaling due to upregulation of TYK2.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)—(XII).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DCE | dichloroethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′-Bis(diphenylphosphino)ferrocene]dichloro- |
| | palladium(II) |
| MeCN (ACN) | Acetonitrile |
| Pd₂(dba)₃ | Tris(dibenzylideneacetone)dipalladium(0) |
| Hunig's Base | N,N-diisopropylethylamine |
| TBAF | Tert butyl ammonium fluoride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| AcOH | Acetic Acid |
| DMAc | N,N-Dimethylformamide |
| BPD | 4,4,5,5-tetramethyl-2-(4,4,5,5-tetramethyl-1,3,2- |
| | dioxaborolan-2-yl)-1,3,2-dioxaborolane |
| MTBE | Methy tert-Butyl Ether |

### Preparation of Intermediate Types A, B, and C

### Example 1: Preparation of tert-butyl N-[3-(4-bromo-1,5-dimethyl-pyrazol-3-yl)oxypropyl]-N-methyl-carbamate (A1)

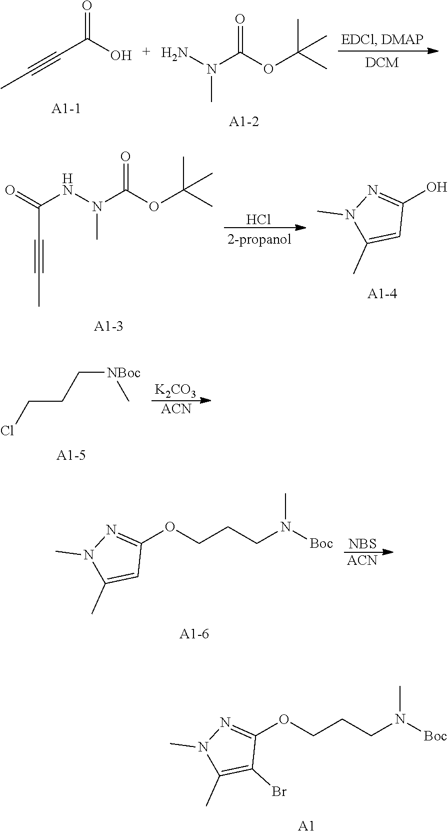

Step 1. To a solution of but-2-ynoic acid (5 g, 59.5 mmol, 1 eq), DMAP (726 mg, 5.95 mmol, 0.1 eq) in DCM (200 mL) was added tert-butyl N-amino-N-methyl-carbamate (9.22 g, 63.0 mmol, 1.06 eq) and EDCI (12.5 g, 65.4 mmol, 1.1 eq) at 0° C. The reaction mixture was stirred at 25° C. for 16 hr. The reaction mixture was concentrated in vacuo to give tert-butyl N-(but-2-ynoylamino)-N-methyl-carbamate (5.5 g, 25.9 mmol, 43.5% yield) as a white oil. ¹H NMR (400 MHz, CDCl₃) δ=7.75 (s, 1H), 3.11 (s, 3H), 1.95 (s, 3H), 1.44 (s, 9H).

Step 2. To a solution of tert-butyl N-(but-2-ynoylamino)-N-methyl-carbamate (5.5 g, 25.9 mmol, 1.00 eq) in 2-propanol (60 mL), hydrogen chloride (4.84 g, 133 mmol, 4.75 mL, 5.13 eq) was added at 25° C., the mixture was stirred for 16 hours at 63° C. under N₂. The reaction mixture was cooled to ambient temperature and concentrated in vacuo. The resulting residue was then charged with CH₃CN (50 mL, 10 mL/g) and concentrated in vacuo. This process was repeated for three times. The final residue was then charged with CH₃CN (20 mL, 4 mL/g) and warmed to 75° C. at 16 h. The resulting slurry was then cooled to ambient temperature over 1 h and allowed to continue to desaturate overnight. The solids were filtered, washed with CH₃CN (2×20 mL), and then dried under vacuum at ambient temperature to afford salt 1,5-dimethylpyrazol-3-ol (5.5 g, 24.5 mmol, 94.6% yield) as a white crystalline solid. ¹H NMR (400 MHz, DMSO-d₆) δ=10.98 (s, 1H), 5.64 (s, 3H), 3.64 (s, 3H), 2.22 (s, 3H).

Step 3. A mixture of 1,5-dimethylpyrazol-3-ol (800 mg, 7.13 mmol, 1 eq), tert-butyl N-(3-chloropropyl)-N-methyl-carbamate (1.63 g, 7.85 mmol, 1.1 eq), K₂CO₃ (1.48 g, 10.7 mmol, 1.5 eq) in DMF (35 mL). The mixture was stirred at 80° C. for 16 h. After cooled to 25° C., the mixture was diluted with water (10 mL), extracted with EA (3×10 mL). The combined organic layer was washed with brine (10 mL), dried over Na₂SO₄, filtered and concentrated in vacuo. The residue was purified by column chromatography on silica gel (PE:EA=30:1-0:1) to give tert-butyl N-[3-(1,5-dimethylpyrazol-3-yl)oxypropyl]-N-methyl-carbamate (600 mg, 2.12 mmol, 29.6% yield) as yellow oil. ¹H NMR (400 MHz, DMSO-d₆) δ=5.41 (s, 1H), 3.97-3.93 (m, 2H), 3.52 (s, 3H), 3.32-3.23 (m, 2H), 2.76 (s, 3H), 2.14 (s, 3H), 1.84-1.80 (m, 2H), 1.36 (s, 9H).

Step 4. To a solution of tert-butyl N-[3-(1,5-dimethylpyrazol-3-yl)oxypropyl]-N-methyl-carbamate (600 mg, 2.12 mmol, 1 eq) in ACN (10 mL) was added NBS (376 mg, 2.12 mmol, 1 eq) at 25° C. for 16 h under N₂. The reaction mixture was concentrated in vacuo. The residue was purified by column chromatography on silica gel (PE:EA=25:1-0:1) to give tert-butyl N-[3-(4-bromo-1,5-dimethyl-pyrazol-3-yl)oxypropyl]-N-methyl-carbamate (550 mg, 1.49 mmol, 70.3% yield, 98% purity) as a yellow solid. ¹H NMR (400 MHz, DMSO-d₆) δ=4.07 (t, J=6.4 Hz, 2H), 3.61 (s, 3H), 3.28 (t, J=6.8 Hz, 2H), 2.77 (s, 3H), 2.16 (s, 3H), 1.87 (t, J=6.4 Hz, 2H), 1.36 (s, 9H).

### Example 2: Preparation of tert-butyl N-[2-[(4-bromo-1,5-dimethyl-pyrazol-3-yl)methoxy]ethyl]-N-methyl-carbamate (A2)

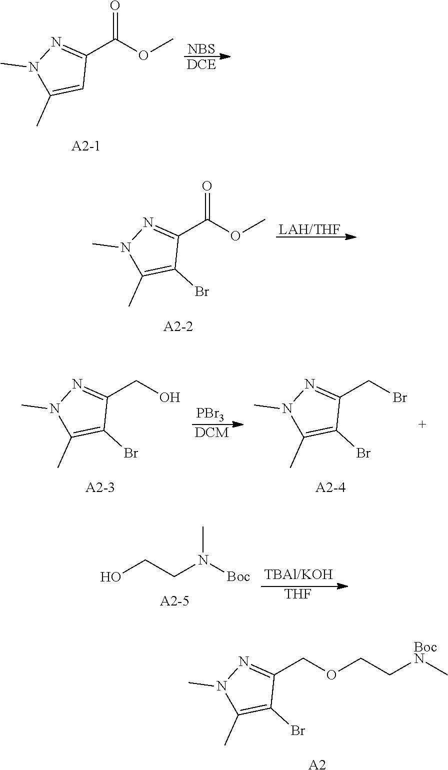

Step 1. To a solution of ethyl 1,5-dimethylpyrazole-3-carboxylate (10.0 g, 59.5 mmol, 1 eq) in DCE (150 mL) was added NBS (15.9 g, 89.2 mmol, 1.5 eq). The mixture was stirred at 80° C. for 16 hours. On completion, the mixture was concentrated in vacuum to give crude. The residue was purified by column chromatography (SiO₂, Petroleum ether/Ethyl acetate=0% to 30%) to give ethyl 4-bromo-1,5-dimethyl-pyrazole-3-carboxylate (7.00 g, 28.3 mmol, 47.7% yield) as white solid. ¹H NMR (400 MHz, CDCl₃) δ=4.43 (t, J=7.2 Hz, 2H), 3.90 (s, 3H), 2.31 (s, 3H), 1.41 (t, J=7.2 Hz, 3H).

Step 2. To a solution of methyl 4-bromo-1,5-dimethyl-pyrazole-3-carboxylate (8.0 g, 34.33 mmol, 1 eq) in THF (100 mL) was added LiAlH₄ (1.56 g, 41.19 mmol, 1.2 eq) at 0° C. The mixture was stirred at 15° C. for 3 h. On completion, the mixture was quenched by H₂O (30 mL) and extracted with EtOAc (20 mL*3) and the resulting organic layer was dried over anhydrous Na₂SO₄, filtered and the filtrate was concentrated in vacuum to give (4-bromo-1,5-dimethyl-pyrazol-3-yl)methanol (4.3 g, 20.97 mmol, 61.09% yield) as white solid. ¹H NMR (400 MHz, DMSO-d6) δ=4.93 (t, J=5.6 Hz, 2H), 4.30-4.27 (m, 2H), 3.72 (s, 3H), 3.32 (s, 3H).

Step 3. To a solution of (4-bromo-1,5-dimethyl-pyrazol-3-yl)methanol (4.30 g, 21.0 mmol, 1 eq) in DCM (40 mL), PBr₃ (5.68 g, 21.0 mmol, 1 eq) was added dropwise at 0° C. The mixture was stirred at 0-25° C. for 4 hours. On completion, the mixture was concentrated in vacuum. It was added NaHCO₃ solution to adjust pH to the value of 7 and extracted with DCM (30 mL×4). The combined organic phase was dried over anhydrous sodium sulfate, filtered and concentrated to give a residue. The residue was purified by combi flash (40 g silica gel column, DCM/MeOH=0 to 20%) to give (3.70 g, 13.8 mmol, 65.9% yield) as a white solid. ¹H NMR (400 MHz, CDCl3) δ=4.51 (s, 3H), 3.80 (s, 3H), 2.50 (s, 3H).

Step 4. To a solution of 4-bromo-3-(bromomethyl)-1,5-dimethyl-pyrazole (4.80 g, 17.9 mmol, 1 eq) in THF (100 mL), tert-butyl N-(2-hydroxyethyl)-N-methyl-carbamate (3.45 g, 19.7 mmol, 63.6 μL, 1.1 eq), TBAI (661 mg, 1.79 mmol, 0.1 eq) and KOH (3.02 g, 53.7 mmol, 3 eq) was added. The mixture was stirred at 25° C. for 16 hours under N₂. On completion, the mixture was concentrated in vacuum. The residue was purified by column chromatography (SiO₂, Petroleum ether/Ethyl acetate=50% to 70%). tert-Butyl N-[2-[(4-bromo-1,5-dimethyl-pyrazol-3-yl)methoxy]ethyl]-N-methyl-carbamate (5.6 g, 15.5 mmol, 86.3% yield) was obtained as white solid. ¹H NMR (400 MHz, CDCl₃) δ=4.43 (s, 2H), 3.78 (s, 3H), 3.60-3.55 (m, 2H), 3.38-3.35 (m, 2H), 2.23 (s, 3H), 1.44 (s, 3H), 1.42 (s, 9H).

### Example 2a: Preparation of tert-butyl N-[(1R)-2-[(4-bromo-1,5-dimethyl-pyrazol-3-yl)methoxy]-1-methyl-ethyl]-N-methyl-carbamate (A2a)

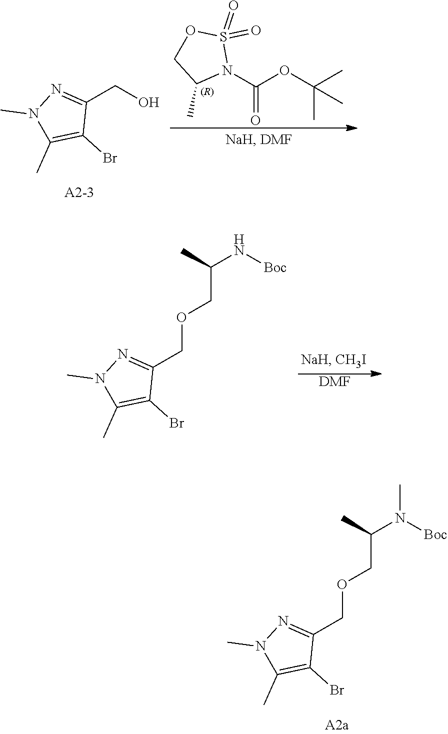

Step 1. To a solution of (4-bromo-1,5-dimethyl-pyrazol-3-yl)methanol (8.64 g, 42.1 mmol, 1 eq) in DMF (180 mL) was added NaH (3.37 g, 84.2 mmol, 60% purity, 2 eq) at 0° C. The mixture was stirred at 25° C. for 0.5 h followed by addition of tertbutyl(4R)-4-methyl-2,2-dioxo-oxathiazolidine-3-carboxylate (15.0 g, 63.2 mmol, 1.5 eq) and stirred at 25° C. for another 1 h. On completion, the mixture was quenched by H₂O (400 mL), extracted with EtOAc (100 mL*3), dried over anhydrous Na₂SO₄, and filtered. The filtrate was concentrated in vacuum and purified by flash silica gel chromatography (80 g silica gel column, MeOH in DCM from 0-10%) to give tert-butyl N-[(1R)-2-[(4-bromo-1,5-dimethyl-pyrazol-3-yl)methoxy]-1-methyl-ethyl]carbamate (10.2 g, 28.1 mmol, 66% yield) as a pink solid. ¹H NMR (400 MHz, DMSO-d₆) δ=6.71 (d, J=8.4 Hz, 1H), 4.36-4.25 (m, 2H), 3.74 (s, 3H), 3.64-3.50 (m, 1H), 3.31-3.27 (m, 1H), 3.19-3.11 (m, 1H), 2.23-2.21 (m, 3H), 1.36 (s, 9H), 0.99 (d, J=6.8 Hz, 3H).

Step 2. To a solution of tert-butyl N-[(1R)-2-[(4-bromo-1,5-dimethyl-pyrazol-3-yl)methoxy]-1-methyl-ethyl]carbamate (5.00 g, 13.8 mmol, 1 eq) in DMF (100 mL) was added NaH (1.10 g, 27.6 mmol, 60% purity, 2 eq) at 0° C. The mixture was stirred at 0° C. for 0.5 h and then CH₃I (2.94 g, 20.7 mmol, 1.29 mL, 1.5 eq) was added. The mixture was stirred at 25° C. for 1 h. On completion, the mixture was quenched by H₂O (50 mL) and extracted with EtOAc (40 mL*3). The combined organic layer was dried over anhydrous Na₂SO₄, filtered and the filtrate was concentrated in vacuum to give crude. The residue was purified by flash silica gel chromatography (40 g silica gel column, THF in PE from 0-50%) to give tert-butyl N—[(1R)-2-[(4-bromo-1,5-dimethyl-pyrazol-3-yl)methoxy]-1-methyl-ethyl]-N-methyl-carbamate (3.35 g, 8.90 mmol, 64% yield). LCMS: m/z 377.7 (M+1).

### Example 3: Preparation of tert-butyl N-[(1R)-2-[(4-bromo-1-methyl-pyrazol-3-yl)methoxy]-1-methyl-ethyl]carbamate (A3)

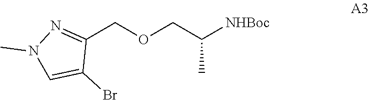 [SMILES: C[C@H](COCc1nn(C)cc1Br)NC(=O)OC(C)(C)C]

A3 was prepared using similar procedures as A2 starting with methyl 1-methylpyrazole-3-carboxylate and tert-butyl N-[(1R)-2-hydroxy-1-methyl-ethyl]carbamate in Step 4 for alkylation reaction. ¹H NMR (400 MHz, DMSO-d₆) δ=7.90 (s, 1H), 6.68-6.44 (m, 2H), 4.33 (d, J=3.6 Hz, 3H), 3.80 (s, 3H), 3.64-3.40 (m, 2H), 1.38-1.36 (m, 9H), 1.00-0.97 (m, 6H).

### Example 4: Preparation of tert-butyl N-[(1R)-2-[(4-bromo-1-methyl-pyrazol-3-yl) methoxy]-1-methyl-ethyl]-N-methyl-carbamate (A4)

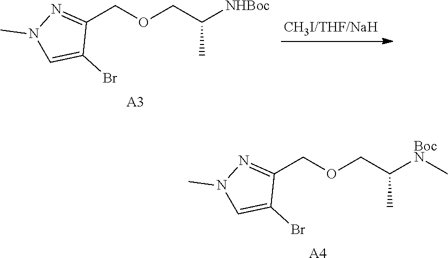

To a solution of tert-butyl N-[(1R)-2-[(4-bromo-1-methyl-pyrazol-3-yl)methoxy]-1-methyl-ethyl]carbamate (A3, 3.50 g, 10.05 mmol, 1.0 eq) in THF (40 mL) was added NaH (1.21 g, 30.2 mmol, 60% purity, 3.0 eq) at 0° C. The mixture was stirred at 0° C. for 0.5 h. And then CH₃I (2.14 g, 15.1 mmol, 939 μL, 1.5 eq) was added. The mixture was stirred at 25° C. for 1.5 h. The mixture was quenched with sat. NH₄Cl(150 mL) and extracted with ethyl acetate (50 mL*3), the combined organic phase was dried over anhydrous sodium sulfate, filtered and concentrated to give tert-butyl N-[(1R)-2-[(4-bromo-1-methyl-pyrazol-3-yl)methoxy]-1-methyl-ethyl]-N-methyl-carbamate (3.20 g, crude) as a colorless oil. LCMS m/z 385.9 (M+23).

### Example 5: Preparation of tert-butyl N-[(1S)-2-[(4-bromo-1-methyl-pyrazol-3-yl) methoxy]-1-methyl-ethyl]-N-methyl-carbamate (A5)

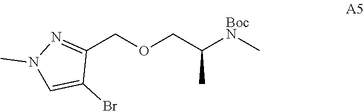

A5 was prepared using similar procedures as A4. LCMS m/z 385.9 (M+23).

### Example 6: Preparation of tert-butyl N-[2-[(4-bromo-1-methyl-pyrazol-3-yl)methoxy]ethyl]-N-methyl-carbamate (A6)

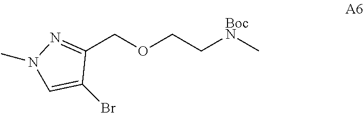

A6 was prepared using similar procedures as A2. LCMS m/z 371.9 (M+23).

### Example 7: Preparation of tert-butyl N-[(1R)-2-[[5-bromo-2-methyl-1-(2-trimethylsilylethoxymethyl)imidazol-4-yl]methoxy]-1-methyl-ethyl]-N-methyl-carbamate (A7)

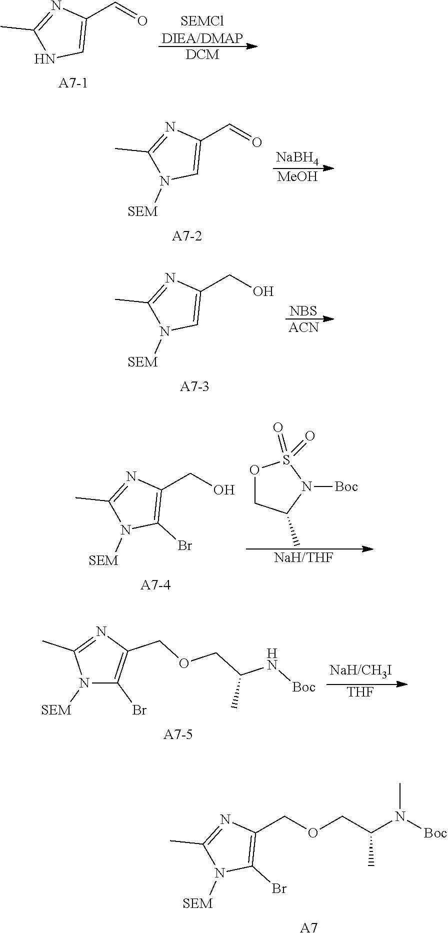

Step 1. To a mixture of 2-methyl-1H-imidazole-4-carbaldehyde (5.00 g, 45.4 mmol, 1 eq) in DCM (50.0 mL) was added SEM-Cl (9.84 g, 59.0 mmol, 10.5 mL, 1.3 eq) at 0° C., followed by DIEA (7.63 g, 59.0 mmol, 10.2 mL, 1.3 eq) and DMAP (277 mg, 2.27 mmol, 0.05 eq), the mixture was stirred at 25° C. for 0.5 hour under N₂. The reaction mixture was quenched by addition MeOH (50 mL) to afford 2-methyl-1-(2-trimethylsilylethoxymethyl)imidazole-4-carbaldehyde (5.00 g, 20.8 mmol, 45.8% yield) as a yellow oil.

Step 2. To a mixture of 2-methyl-1-(2-trimethylsilylethoxymethyl)imidazole-4-carbaldehyde (5.00 g, 20.8 mmol, 1 eq) in MeOH (50.0 mL) was added NaBH₄ (825 mg, 21.8 mmol, 1.05 eq) at 0° C. The mixture was stirred at 25° C. for 0.5 hour under N₂ and then quenched by addition of water (20.0 mL) followed by extraction with EtOAc (20.0 mL*3). The combined organic layers were dried over Na₂SO₄, filtered and concentrated under reduced pressure to give a crude residue. The residue was purified by flash silica gel chromatography (ISCO®; 40.0 g SepaFlash® Silica Flash Column, Eluent of 0˜6% Ethylacetate/Petroleum ether gradient @50 mL/min) to afford [2-methyl-1-(2-trimethylsilylethoxymethyl)imidazol-4-yl]methanol (5.00 g, 18.46 mmol, 88.76% yield, 89.5% purity) as a yellow oil. LCMS m/z 243.3 (M+1).

Step 3. To a mixture of [2-methyl-1-(2-trimethylsilylethoxymethyl)imidazol-4-yl]methanol (5.00 g, 20.6 mmol, 1 eq) in ACN (50.0 mL) was added NBS (4.41 g, 24.7 mmol, 1.2 eq) at 0° C. The mixture was stirred at 25° C. for 1 hour under N₂ and then partitioned between EtOAc (20.0 mL) and H₂O (20.0 mL). The organic phase was separated, washed with brine (10.0 mL*3), dried over Na₂SO₄, filtered and concentrated under reduced pressure to give a residue. The residue was purified by flash silica gel chromatography (ISCO®; 20 g SepaFlash® Silica Flash Column, Eluent of 0-20% Ethylacetate/Petroleum ether gradient @60 mL/min) to afford [5-bromo-2-methyl-1-(2-trimethylsilylethoxymethyl)imidazol-4-yl]methanol (5.00 g, 14.78 mmol, 71.67% yield, 95.0% purity) as a yellow oil. LCMS m/z 323.1 (M+1).

Step 4. To a mixture of [5-bromo-2-methyl-1-(2-trimethylsilylethoxymethyl)-imidazol-4-yl]methanol (500 mg, 1.56 mmol, 1 eq) in THF (10.0 mL) was added NaH (93.3 mg, 2.33 mmol, 60% purity, 1.5 eq) at 0° C. The mixture was stirred at 0° C. for 0.5 hour. Then tert-butyl (4R)-4-methyl-2,2-dioxo-oxathiazolidine-3-carboxylate (443 mg, 1.87 mmol, 1.2 eq) was added, the mixture was stirred at 25° C. for 1.5 hours. The reaction mixture was quenched by addition of H₂O (5.00 mL). The filter liquor was diluted with H₂O (5.00 mL) and extracted with EtOAc (5.00 mL*3). The combined organic layers were washed with brine (5 mL*3), dried over Na₂SO₄, filtered and concentrated under reduced pressure. The residue was purified by prep-TLC (SiO₂, Petroleum ether:Ethyl acetate=0:1) to give tert-butyl N-[(1R)-2-[[5-bromo-2-methyl-1-(2-trimethylsilylethoxymethyl)imidazol-4-yl]methoxy]-1-methyl-ethyl]carbamate (600 mg, 1.17 mmol, 75.1% yield) as a yellow oil. LCMS 479.5 (M+1).

Step 5. To a mixture of tert-butyl N-[(1R)-2-[[5-bromo-2-methyl-1-(2-trimethylsilylethoxymethyl)imidazol-4-yl]methoxy]-1-methyl-ethyl]carbamate (600 mg, 1.25 mmol, 1 eq) in THF (7.00 mL) was added NaH (75.2 mg, 1.88 mmol, 60% purity, 1.5 eq) at 0° C., the mixture was stirred at 0° C. for 0.5 hour. Then CH₃I (267 mg, 1.88 mmol, 117 μL, 1.5 eq) was added, and the mixture was stirred at 25° C. for 1.5 hours. The reaction mixture was quenched by addition of H₂O (5.00 mL) at 25° C. The filter liquor was diluted with H₂O (5.00 mL) and extracted with EtOAc (5.00 mL*3). The combined organic layers were washed with brine (5.00 mL*3), dried over Na₂SO₄, filtered and concentrated under reduced pressure to afford tert-butyl N-[(1R)-2-[[5-bromo-2-methyl-1-(2-trimethylsilylethoxymethyl)imidazol-4-yl]methoxy]-1-methyl-ethyl]-N-methyl-carbamate (550 mg) as a yellow oil. LCMS 494.1 (M+1: 494.1).

### Example 8: Preparation of [3-[[(2R)-2-[tert-butoxycarbonyl(methyl)amino]propoxy]methyl]-1-methyl-pyrazol-4-yl]boronic acid (B1)

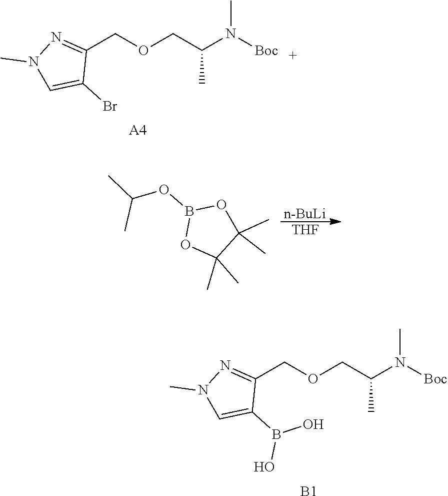

To a solution of tert-butyl N-[(1R)-2-[(4-bromo-1-methyl-pyrazol-3-yl)methoxy]-1-methyl-ethyl]-N-methyl-carbamate (2.00 g, 5.52 mmol, 1.0 eq) in THF (20 mL) was added n-BuLi (2.5 M, 5.52 mL, 2.5 eq). The mixture was stirred at −70° C. for 1 hr and then 2-isopropoxy-4,4,5,5-tetramethyl-1,3,2-dioxaborolane (3.08 g, 16.6 mmol, 3.38 mL, 3.0 eq) was added at −70° C. The mixture was stirred at −70° C. for 3 hr. The mixture was quenched with water (50 mL) and extracted with ethyl acetate (25 mL×3), the combined aqueous phase was dried over anhydrous sodium sulfate, filtered and concentrated to give [3-[[(2R)-2-[tert-butoxycarbonyl(methyl)amino]propoxy]methyl]-1-methyl-pyrazol-4-yl]boronic acid (1.75 g, crude) was obtained as a white solid. LCMS m/z 327.7 (M+1).

### Example 8b: Preparation of 7-fluoro-5-(4,4,5,5-tetramethyl-1,3,2-dioxaborolan-2-l)indolin-2-one (D1)

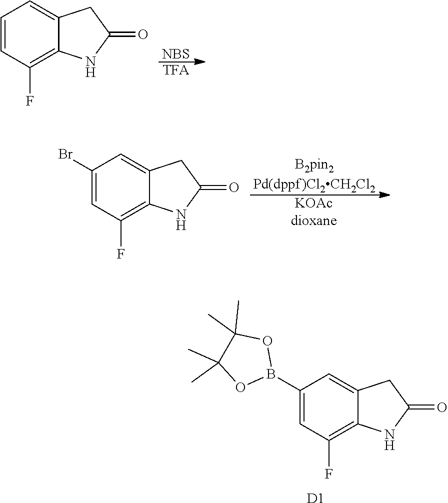

Step 1. To a solution of 7-fluoroindolin-2-one (10.0 g, 66.1 mmol, 1 eq) in TFA (100 mL) was added NBS (12.9 g, 72.7 mmol, 1.1 eq) at 0° C. and stirred for 0.5 h. The mixture was the stirred at 25° C. for 6 h. On completion, the reaction mixture was concentrated under reduced pressure to remove solvent. The crude product was triturated with DCM:PE=(1:1, 400 mL) at 20° C. for 5 min to give 5-bromo-7-fluoro-indolin-2-one (15.4 g, 59.7 mmol, 90% yield) as a brown solid. ¹H NMR (400 MHz, DMSO-d₆) δ=11.05 (s, 1H), 7.41 (dd, J=9.6 Hz, 1H), 7.27 (s, 1H), 3.58 (s, 2H).

Step 2. To a solution of 5-bromo-7-fluoro-indolin-2-one (16.8 g, 73.2 mmol, 1 eq) and 4,4,5,5-tetramethyl-2-(4,4,5,5-tetramethyl-1,3,2-dioxaborolan-2-yl)-1,3,2-dioxaborolane (55.8 g, 219 mmol, 3 eq) in dioxane (250 mL) was added potassium acetate (28.7 g, 292 mmol, 4 eq) and cyclopentyl(diphenyl)phosphane; dichloromethane; dichloropalladium; iron (5.98 g, 7.32 mmol, 0.1 eq). The mixture was stirred at 100° C. for 16 h under N₂. On completion, the reaction mixture was concentrated under reduced pressure to remove solvent. The residue was purified by flash silica gel chromatography (ISCO®; 220 g SepaFlash® Silica Flash Column, Eluent of 0-20% MeOH/DCM @40 mL/min) to give 7-fluoro-5-(4,4,5,5-tetramethyl-1,3,2-dioxaborolan-2-yl)indolin-2-one (14.3 g, 42.9 mmol, 58% yield) as a yellow solid. ¹H NMR (400 MHz, DMSO-d₆) δ=11.01 (s, 1H), 7.32 (s, 1H), 7.24 (d, J=10.4 Hz, 1H), 3.56 (s, 2H), 1.27 (s, 12H).

### Example 9: Preparation of 5-formyl-2-methyl-3-(2-trimethylsilylethoxymethyl)-imidazole-4-carboxylic acid (Cl)

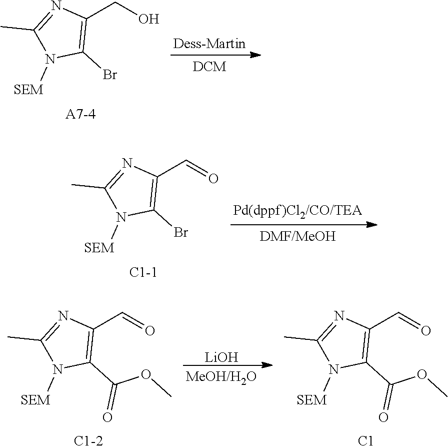

Step 1. To a mixture of [5-bromo-2-methyl-1-(2-trimethylsilylethoxymethyl)-imidazol-4-yl]methanol (1.50 g, 4.67 mmol, 1 eq) in DCM (15.0 mL) at 0° C. was added Dess-Martin reagent (2.97 g, 7.00 mmol, 2.17 mL, 1.5 eq). The mixture was stirred at 25° C. for 2 hours, diluted with DCM (10.0 mL), and filtered to remove the insoluble. The filter liquor was diluted with H₂O (10.0 mL) and extracted with DCM (10.0 mL*3). The combined organic layers were washed with brine (10.0 mL*3), dried over Na₂SO₄, filtered and concentrated under reduced pressure. The residue was purified by flash silica gel chromatography (ISCO®; 10 g SepaFlash® Silica Flash Column, Eluent of 0-10% Ethylacetate/Petroleum ether gradient @55 mL/min) to afford 5-bromo-2-methyl-1-(2-trimethylsilylethoxymethyl)-imidazole-4-carbaldehyde (710 mg, 2.22 mmol, 47.63% yield) as a colorless oil. ¹H NMR (400 MHz, Chloroform-d) 6=9.60 (s, 1H), 5.75-5.68 (m, 2H), 3.62-3.53 (m, 2H), 2.55-2.46 (m, 3H), 0.94-0.84 (m, 2H), −0.02 (s, 9H).

Step 2. To a solution of 5-bromo-2-methyl-1-(2-trimethylsilylethoxymethyl)imidazole-4-carbaldehyde (700 mg, 2.19 mmol, 1eq) in DMF (10.0 mL) and MeOH (5.00 mL) were added Pd(dppf)Cl₂ (80.2 mg, 109 μmol, 0.05 eq) and TEA (665 mg, 6.58 mmol, 915 μL, 3 eq) under N₂. The mixture was degassed under vacuum and purged with CO several times. The mixture was stirred under CO (2.19 mmol, 1 eq) (50 psi) at 80° C. for 16 hours. The mixture was concentrated in vacuum. The crude product was purified by silica gel chromatography eluted with Petroleum ether/Ethylacetate=5:1 to afford methyl 5-formyl-2-methyl-3-(2-trimethylsilylethoxymethyl)imidazole-4-carboxylate (550 mg, 1.84 mmol, 84.06% yield) as a yellow solid. ¹H NMR (400 MHz, CDCl₃) δ=10.4 (s, 1H), 5.78 (s, 2H), 3.98 (s, 3H), 3.64-3.47 (m, 2H), 2.56 (s, 3H), 0.92-0.86 (m, 2H), −0.03 (s, 9H).

Step 3. To a mixture of methyl 5-formyl-2-methyl-3-(2-trimethylsilylethoxymethyl)-imidazole-4-carboxylate (300 mg, 1.01 mmol, 1 eq) in MeOH (2.00 mL) and H₂O (0.6 mL) was added LiOH·H₂O (210 mg, 5.03 mmol, 5 eq) under N₂. The mixture was stirred at 25° C. for 1 hour. The reaction mixture was concentrated under reduced pressure to remove MeOH. To the mixture was added HCl (1 N) until pH=5-6. The residue was diluted with H₂O (5.00 mL) and extracted with EtOAc (5.00 mL*3). The combined organic layers were washed with brine (5.00 mL*3), dried over Na₂SO₄, filtered and concentrated under reduced pressure to give a residue to afford 5-formyl-2-methyl-3-(2-trimethylsilylethoxymethyl)imidazole-4-carboxylic acid (200 mg, 694 μmol, 69.12% yield, 98.8% purity) as a white solid. ¹H NMR (400 MHz, DMSO-d₆) δ=10.31 (s, 1H), 5.70 (s, 2H), 3.53-3.49 (m, 2H), 2.45 (s, 3H), 0.89-0.78 (m, 3H), −0.05-−0.07 (m, 9H).

### General Method A

### Example 9: Preparation of [3a(4)Z]-6,9,15,16-tetramethyl-9,10,11,12-tetrahydro-15H-1,17-(ethanediylidene)pyrazolo[4,3-n]dipyrrolo[3,2-g:3′,4′-j][1,5]oxazacyclopentadecine-3,8(2H,5H)-dione (Cpd. 1)

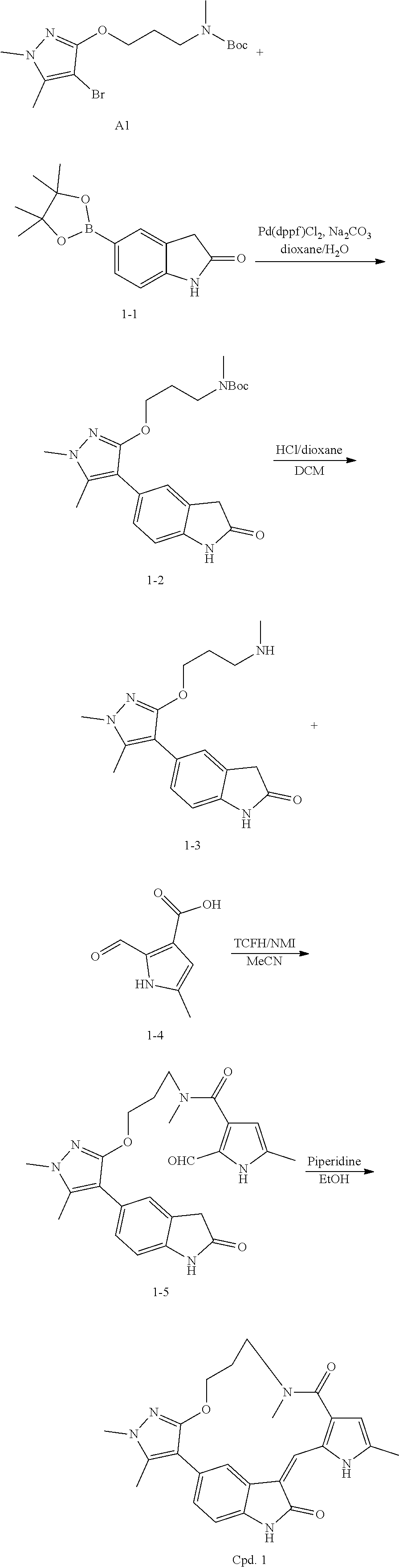

Step 1. To a solution of tert-butyl N-[3-(4-bromo-1,5-dimethyl-pyrazol-3-yl)oxypropyl]-N-methyl-carbamate (500 mg, 1.38 mmol, 1 eq), 5-(4,4,5,5-tetramethyl-1,3,2-dioxaborolan-2-yl)indolin-2-one (643.73 mg, 2.48 mmol, 1.8 eq), Na₂CO₃ (438 mg, 4.14 mmol, 3 eq) in dioxane (20 mL) and H₂O (4 mL) was added Pd(dppf)Cl₂ (100 mg, 138 μmol, 0.1 eq). The reaction mixture was stirred at 100° C. for 2 hr, cooled and concentrated in vacuo. The residue was purified by column chromatography on silica gel (DCM:MeOH=25:1-10:1) to give tert-butyl N-[3-[1,5-dimethyl-4-(2-oxoindolin-5-yl)pyrazol-3-yl]oxypropyl]-N-methyl-carbamate (450 mg, 662 μmol, 47.9% yield, 61% purity) as a yellow oil. LCMS m/z 415.2 ((M+1).

Step 2. To a solution of 5-[1,5-dimethyl-3-[3-(methylamino)propoxy]pyrazol-4-yl]indolin-2-one (160 mg, 508 μmol, 1 eq), 2-formyl-5-methyl-1H-pyrrole-3-carboxylic acid (116.90 mg, 763.40 μmol, 1.5 eq), 1-methylimidazole (417 mg, 5.09 mmol, 0.040 mL, 10 eq) and [chloro(dimethylamino)methylene]-dimethyl-ammonium; hexafluorophosphate (185 mg, 662 μmol, 1.3 eq) in MeCN (30 mL) was stirred at 25° C. for 1 h. The reaction mixture was concentrated in vacuo. The residue was purified by column chromatography on silica gel (DCM:MeOH=30:1-10:1) to give N-[3-[1,5-dimethyl-4-(2-oxoindolin-5-yl)pyrazol-3-yl]oxypropyl]-2-formyl-N,5-dimethyl-1H-pyrrole-3-carboxamide (50 mg, 0.0745 mmol, 14.6% yield, 67% purity) as a yellow solid. LCMS m/z 450.2 (M+1).

Step 3. To a mixture of N-[3-[1,5-dimethyl-4-(2-oxoindolin-5-yl)pyrazol-3-yl]oxypropyl]-2-formyl-N,5-dimethyl-1H-pyrrole-3-carboxamide (50 mg, 0.111 mmol, 1 eq) in EtOH (10 mL) was added piperidine (14.2 mg, 0.167 mmol, 1.5 eq) at 25° C. The mixture was stirred at 80° C. for 1 hr and concentrated in vacuo. The residue was purified by column chromatography on silica gel (DCM:MeOH=30:1-10:1) to give Cpd. 1 (2.9 mg, 5.86% yield, 97% purity) as orange solid

### Example 10

Cpd. 2-Cpd. 8 were prepared using General Method A with corresponding bromo pyrazole analog A1, A2, A3, A4, A5 or A6, commercially available oxindole boronic ester 5-(4,4,5,5-tetramethyl-1,3,2-dioxaborolan-2-yl)indolin-2-one or 7-chloro-5-(4,4,5,5-tetramethyl-1,3,2-dioxaborolan-2-yl)indolin-2-one, and pyrrole or imidazole aldehyde acid 2-formyl-5-methyl-1H-pyrrole-3-carboxylic acid, 2-formyl-1H-pyrrole-3-carboxylic acid, or 5-formyl-2-methyl-3-(2-trimethylsilylethoxymethyl)imidazole-4-carboxylic acid.

Cpd. 8 was obtained with an addition deprotection step: To a mixture of SEM-protected Cpd. 8 on imidazole ring (40.0 mg, 0.067.0 mmol, 1 eq) in DCM (3 mL) was added TFA (1.54 g, 13.5 mmol, 1 mL, 201 eq). The mixture was stirred at 25° C. for 1 hour and concentrated under reduced pressure. The residue was purified by prep-HPLC (column: Phenomenex C18 150*25 mm*10 μm; mobile phase: [water(NH₄HCO₃)-ACN]; B %: 9%-39%, 8 min) to afford Cpd. 8 (5.48 mg, 0.012 mmol) as a yellow solid.

| Cpd # | Structure | ¹H NMR (400 MHz, DMSO-d₆) δ ppm | MS m/z |
|---|---|---|---|
| 1 | 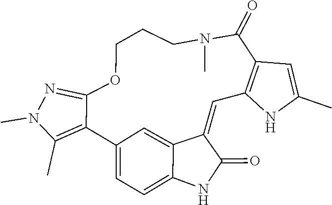 | 11.45 (s, 1H), 10.72 (s, 1H), 7.87 (s, 1H), 7.29 (s, 1H), 7.20-7.16 (m, 1H), 6.87 (d, J = 8.0 Hz, 1H), 6.20 (d, J = 2.0 Hz, 1H), 4.34-4.30 (m, 2H), 3.66 (s, 3H), 3.32 (s, 2H), 2.92 (s, 3H), 2.39 (s, 3H), 2.38 (s, 3H), 2.08-1.92 (m, 2H) | 432.1 (M + 1)⁺ |
| 2 | 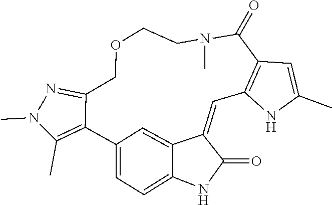 | 11.44 (s, 1H), 10.76 (s, 1H), 8.13 (s, 1H), 7.49 (s, 1H), 7.20 (d, J = 8.0 Hz, 1H), 6.92 (d, J = 8.0 Hz, 1H), 6.24 (d, J = 1.6 Hz, 1H), 4.61-4.49 (m, 1H), 4.48-4.30 (m, 1H), 4.29-4.26 (m, 1H), 3.95-3.90 (m, 2H), 3.80 (s, 2H), 3.10-3.03 (m, 1H), 3.02 (s, 3H), 2.33 (s, 6H) | 432.3 (M + 1)⁺ |
| 3 | 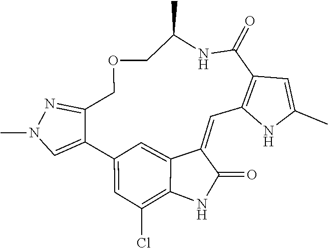 | 11.61 (s, 1H), 11.18 (s, 1H), 8.47-8.46 (m, 1H), 8.22 (s, 1H), 8.12 (s, 1H), 8.06 (s, 1H), 7.28 (d, J = 1.2 Hz, 1H), 6.33 (d, J = 2.0 Hz, 1H), 4.43-4.36 (m, 2H), 4.21- 4.19 (m, 1H), 3.84 (s, 3H), 3.73-3.71 (m, 2H), 3.30 (d, J = 8.0 Hz, 1H), 2.49 (s, 3H), 1.23-1.18 (m, 3H) | 452.1 (M + 1)⁺ |
| 4 | 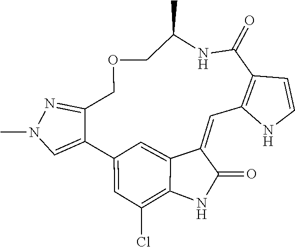 [SMILES: C[C@@H]1COCc2nn(C)cc2-c2cc(Cl)c3c(c2)/C(=C/c2[nH]ccc2C(=O)N1)C(=O)N3] | 11.81 (s, 1H), 11.22 (s, 1H), 8.51-8.49 (m, 1H), 8.25-8.20 (m, 2H), 8.12 (s, 1H), 8.06 (s, 1H), 7.37-7.31 (m, 1H), 6.54 (d, J = 2.0 Hz, 1H),4.43-4.36 (m, 2H), 4.21- 4.19 (m, 1H), 3.84 (s, 3H), 3.73-3.71 (m, 2H), 3.32 (s, 1H), 1.21-1.20 (m, 1H). | 438.1 (M + 1)⁺ |
| 5 | 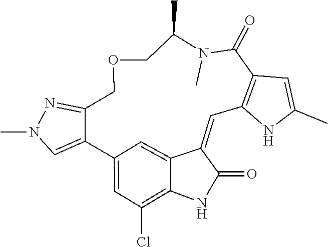 | 11.41 (s, 1H), 11.19 (s, 1H), 8.08 (d, J = 1.2 Hz, 1H), 8.03 (s, 1H), 7.42 (s, 1H), 7.27 (d, J = 1.2 Hz, 1H), 6.27 (d, J = 2.0 Hz, 1H), 4.73-4.69 (m, 1H), 4.43 (q, J = 21.6, 11.2 Hz, 2H), 3.83 (s, 3H), 3.88 - 3.82 (m, 2H), 2.77 (s, 3H), 2.41 (s, 3H), 1.19 (d, J = 7.2 Hz, 3H). | 466.0 (M + 1)⁺ |
| 6 | 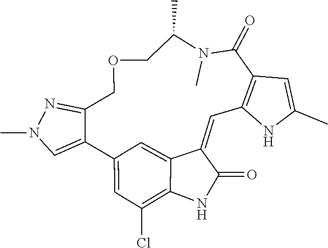 | 11.41 (s, 1H), 11.19 (s, 1H), 8.10 (s, 1H), 8.03 (s, 1H), 7.43 (s, 1H), 7.28 (s, 1H), 6.28 (s, 1H), 4.74-4.69 (m, 1H), 4.44 (q, J = 11.2Hz, 2H), 3.84 (s, 3H), 3.79 (br d, J = 5.8 Hz, 2H), 2.77 (s, 3H), 2.41 (s, 3H), 1.19 (d, J = 6.8 Hz, 3H). | 466.0 (M + 1)⁺ |
| 7 | 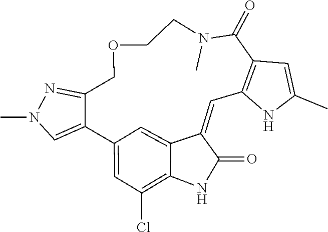 | 11.40 (s, 1H), 8.19-8.04 (m, 2H), 7.47 (s, 1H), 7.34 (d, J = 1.2 Hz, 1H), 6.28 (s, 1H), 4.50-4.40 (m, 3H), 3.83 (s, 3H), 3.08-2.99 (m, 2H), 2.94 (s, 3H), 2.41 (s, 3H). | 452.0 (M + 1)⁺ |
| 8 | 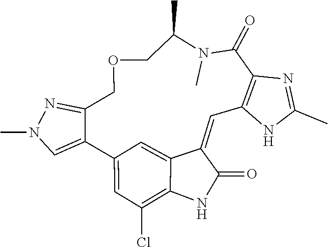 | 12.70 (s, 1H), 11.00-10.86 (m, 1H), 8.11- 8.00 (m, 1H), 7.79 (s, 1H), 7.52-7.40 (m, 1H), 7.30 (d, J = 1.6 Hz, 1H), 5.78- 5.60 (m, 1H), 4.39-4.24 (m, 1H), 4.16- 4.08 (m, 1H), 3.88-3.80 (m, 3H), 3.39 (s, 1H), 3.17-3.12 (m, 1H), 2.38 (s, 3H), 2.28-2.23 (m, 3H), 1.14 (d, J = 7.2 Hz, 3H). | 467.0 (M + 1)⁺ |

### Example 11: Preparation of [3a(4)Z,10R]-20-chloro-6,9,10,15-tetramethyl-10,11,13,16-tetrahydro-2H-1,17-(ethanediylidene)imidazol[4,5-m]dipyrrolo[3,2-f 3′,4′-i][1,4]oxazacyclopentadecine-3,8(5H,9H1)-dione (Cpd. 9)

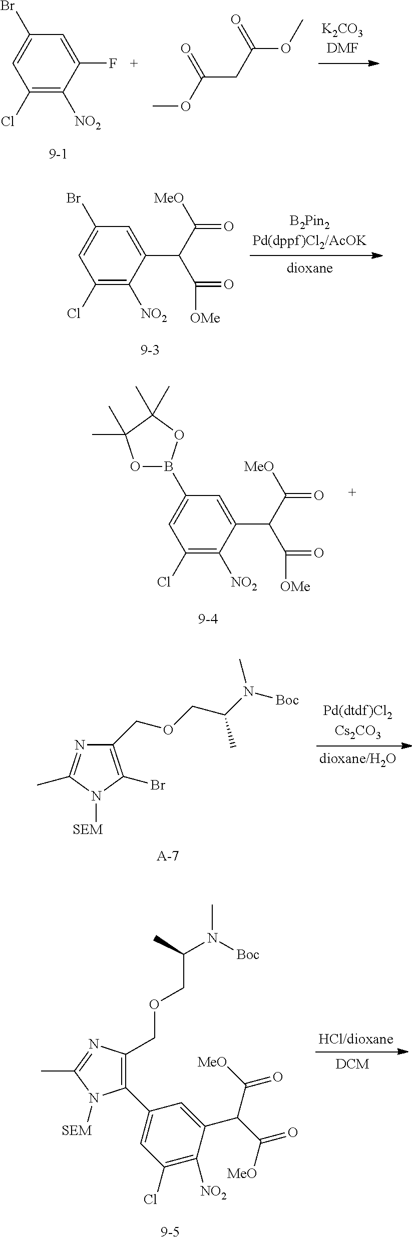

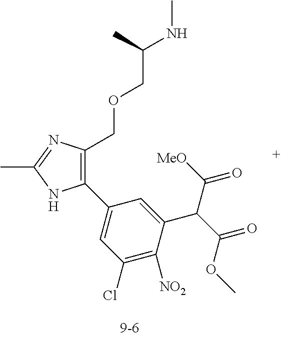 [SMILES: CN[C@H](C)COCc1nc(C)[nH]c1-c1cc(Cl)c([N+](=O)[O-])c(C(C(=O)OC)C(=O)OC)c1]

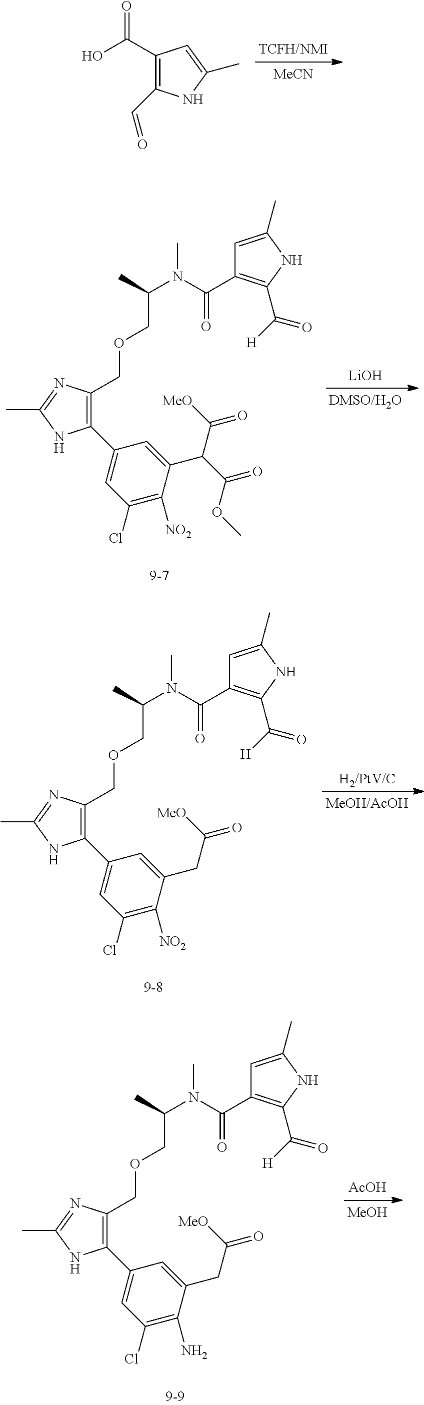

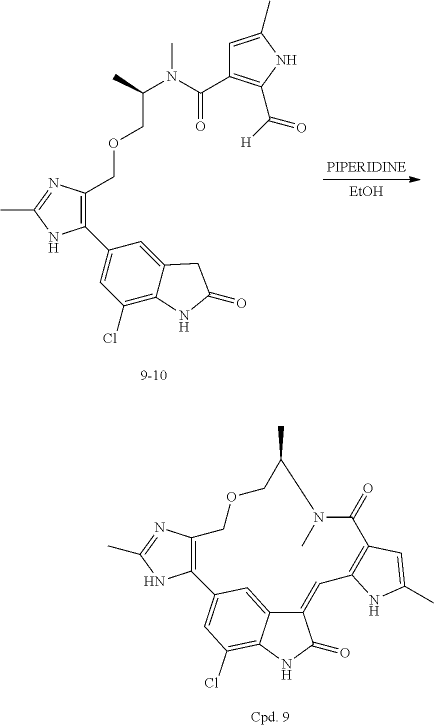

Step 1. To a mixture of 5-bromo-1-chloro-3-fluoro-2-nitro-benzene (2.75 g, 10.8 mmol, 1 eq) and dimethyl propanedioate (1.86 g, 14.0 mmol, 1.61 mL, 1.3 eq) in DMF (30 mL) was added K₂CO₃ (2.99 g, 21.6 mmol, 2 eq) under N₂. The mixture was stirred at 80° C. for 12 h. The residue was diluted with H₂O (15.0 mL) and extracted with EtOAc (15.0 mL*3). The combined organic layers were washed with brine (15 mL*3), dried over Na₂SO₄, filtered and concentrated under reduced pressure to give a residue. The residue was purified by flash silica gel chromatography (ISCO®; 20 g SepaFlash® Silica Flash Column, Eluent of 0-16% Ethylacetate/Petroleum ether gradient @80 mL/min) to afford dimethyl 2-(5-bromo-3-chloro-2-nitro-phenyl)propanedioate (3.20 g, crude) was obtained as a yellow solid. LCMS: m/z 367.7 (M+1)

Step 2. To a mixture of dimethyl 2-(5-bromo-3-chloro-2-nitro-phenyl)propanedioate (3.1 g, 8.46 mmol, 1 eq) and 4,4,5,5-tetramethyl-2-(4,4,5,5-tetramethyl-1,3,2-dioxaborolan-2-yl)-1,3,2-dioxaborolane (2.36 g, 9.30 mmol, 1.1 eq) in dioxane (35.0 mL) was added Pd(dppf)Cl₂ (619 mg, 845 μmol, 0.1 eq) and KOAc (2.49 g, 25.3 mmol, 3 eq) under N₂. The mixture was stirred at 80° C. for 5 h. The reaction mixture was concentrated under reduced pressure to remove dioxane. The residue was purified by column chromatography (SiO₂, Petroleum ether/Ethyl acetate=1/0 to 5/1) to afford compound dimethyl 2-[3-chloro-2-nitro-5-(4,4,5,5-tetramethyl-1,3,2-dioxaborolan-2-yl)phenyl]propanedioate (2.00 g, 4.84 mmol, 57.17% yield) was obtained as a yellow solid. ¹H NMR (400 MHz, DMSO-d₆) δ=7.90 (d, J=0.8 Hz, 1H), 7.81 (d, J=0.8 Hz, 1H), 5.10 (s, 1H), 3.71 (s, 6H), 1.32 (s, 12H).

Step 3. To a mixture of dimethyl 2-[3-chloro-2-nitro-5-(4,4,5,5-tetramethyl-1,3,2-dioxaborolan-2-yl)phenyl]propanedioate (295 mg, 0.714 mmol, 1.1 eq) and tert-butyl N-[(1R)-2-[[5-bromo-2-methyl-1-(2-trimethylsilylethoxymethyl)imidazol-4-yl]methoxy]-1-methyl-ethyl]-N-methyl-carbamate (320 mg, 0.649 mmol, 1 eq) in dioxane (5.00 mL) and H₂O (0.8 mL) was added ditert-butyl(cyclopentyl)phosphane; dichloropalladium; iron (42.3 mg, 64.9 μmol, 0.1 eq) and Cs₂CO₃ (635 mg, 1.95 mmol, 3 eq) under N₂. The mixture was stirred at 70° C. for 2 h. The reaction mixture was concentrated under reduced pressure to remove dioxane. The residue was purified by prep-TLC (SiO₂, Petroleum ether:Ethyl acetate=1:1) to give dimethyl 2-[5-[5-[[(2R)-2-[tert-butoxycarbonyl(methyl)amino]propoxy]methyl]-2-methyl-3-(2-trimethylsilylethoxymethyl)imidazol-4-yl]-3-chloro-2-nitro-phenyl]propanedioate (250 mg, 0.312 mmol, 48.09% yield) as a colorless oil. LCMS: m/z 699.3 (M+1).

Step 4. To a mixture of dimethyl 2-[5-[5-[[(2R)-2-[tert-butoxycarbonyl(methyl)amino]propoxy]methyl]-2-methyl-3-(2-trimethylsilylethoxy methyl)imidazol-4-yl]-3-chloro-2-nitro-phenyl]propanedioate (100 mg, 0.143 mmol, 1 eq) in DCM (2.00 mL) was added HCl/dioxane (4 M, 2.50 mL, 69.9 eq). The mixture was stirred at 20° C. for 1 hour. The reaction mixture was concentrated under reduced pressure to remove DCM to afford dimethyl 2-[3-chloro-5-[2-methyl-4-[[(2R)-2-(methylamino)propoxy]methyl]-1H-imidazol-5-yl]-2-nitro-phenyl]propanedioate (80.0 mg) was obtained as a yellow solid. LCMS m/z 469.0 (M+1)

Step 5. To a mixture of dimethyl 2-[3-chloro-5-[2-methyl-4-[[(2R)-2-(methylamino)propoxy]methyl]-1H-imidazol-5-yl]-2-nitro-phenyl]propanedioate (80.0 mg, 0.158 mmol, 1 eq, HCl) and 2-formyl-5-methyl-1H-pyrrole-3-carboxylic acid (36.3 mg, 0.237 mmol, 1.5 eq) in MeCN (1.50 mL) was added [chloro(dimethylamino)methylene]-dimethyl-ammonium; hexafluorophosphate (66.6 mg, 0.237 mmol, 1.5 eq) and 1-methylimidazole (38.9 mg, 0.475 mmol, 3 eq), the mixture was stirred at 25° C. for 0.5 hour. The reaction mixture was concentrated under reduced pressure to remove DCM. The residue was purified by prep-TLC (SiO₂, Dichloromethane:Methanol=10:1) to afford dimethyl 2-[3-chloro-5-[4-[[(2R)-2-[(2-formyl-5-methyl-1H-pyrrole-3-carbonyl)-methyl-amino]propoxy]methyl]-2-methyl-1H-imidazol-5-yl]-2-nitro-phenyl]propanedioate (80.0 mg, 0.114 mmol, 72.45% yield) as a yellow oil. LCMS m/z 604.3 (M+1).

Step 6. To a mixture of dimethyl 2-[3-chloro-5-[4-[[(2R)-2-[(2-formyl-5-methyl-1H-pyrrole-3-carbonyl)-methyl-amino]propoxy]methyl]-2-methyl-1H-imidazol-5-yl]-2-nitro-phenyl]propanedioate (80.0 mg, 0.132 μmol, 1 eq) in DMSO (1.50 mL) and H₂O (0.3 mL) was added LiCl (22.4 mg, 0.529 mmol, 4 eq). The mixture was stirred at 100° C. for 12 hours. The filter liquor was diluted with H₂O (5.00 mL) and extracted with EtOAc (5.00 mL*3). The combined organic layers were washed with brine (5.00 mL*3), dried over Na₂SO₄, filtered and concentrated under reduced pressure to give a residue. The residue was purified by prep-TLC (SiO₂, Dichloromethane:Methanol=10:1) to afford methyl 2-[3-chloro-5-[4-[[(2R)-2-[(2-formyl-5-methyl-1H-pyrrole-3-carbonyl)-methyl-amino]propoxy]methyl]-2-methyl-1H-imidazol-5-yl]-2-nitro-phenyl]acetate (70.0 mg, crude) as a yellow oil. LCMS m/z 546.0 (M+1).

Step 7. To a solution of methyl 2-[3-chloro-5-[4-[[(2R)-2-[(2-formyl-5-methyl-1H-pyrrole-3-carbonyl)-methyl-amino]propoxy]methyl]-2-methyl-1H-imidazol-5-yl]-2-nitro-phenyl]acetate (40.0 mg, 0.0732 mmol, 1 eq) in MeOH (6.00 mL) and AcOH (0.100 mL) was added Pt/V/C (1.91 mg, 7.33 μmol, 0.1 eq) under N₂. The suspension was degassed under vacuum and purged with H₂ several times. The mixture was stirred under H₂ (15 psi) at 25° C. for 2 hours. The reaction mixture was filtered and the filtrate was concentrated to afford methyl 2-[2-amino-3-chloro-5-[4-[[(2R)-2-[(2-formyl-5-methyl-1H-pyrrole-3-carbonyl)-methyl-amino]propoxy]methyl]-2-methyl-1H-imidazol-5-yl]phenyl]acetate (35.0 mg, crude) as a yellow oil. LCMS m/z 516.2 (M+1).

Step 8. To a mixture of methyl 2-[2-amino-3-chloro-5-[4-[[(2R)-2-[(2-formyl-5-methyl-1H-pyrrole-3-carbonyl)-methyl-amino]propoxy]methyl]-2-methyl-1H-imidazol-5-yl]phenyl]acetate (35.0 mg, 67.8 μmol, 1 eq) in MeOH (2.00 mL) was added AcOH (0.4 mg, 0.0068 mmol, 0.1 eq), the mixture was stirred at 70° C. for 12 hours. The reaction mixture was concentrated under reduced pressure to remove MeOH to afford compound N-[(1R)-2-[[5-(7-chloro-2-oxo-indolin-5-yl)-2-methyl-1H-imidazol-4-yl]methoxy]-1-methyl-ethyl]-2-formyl-N,5-dimethyl-1H-pyrrole-3-carboxamide (25.0 mg, crude) as a yellow oil. LCMS m/z 484.2 (M+1).

Step 9. To a mixture of N-[(1R)-2-[[5-(7-chloro-2-oxo-indolin-5-yl)-2-methyl-1H-imidazol-4-yl]methoxy]-1-methyl-ethyl]-2-formyl-N,5-dimethyl-1H-pyrrole-3-carboxamide (25.0 mg, 0.052 mmol, 1 eq) in EtOH (2.00 mL) was added Piperidine (0.44 mg, 0.0052 mmol, 0.1 eq). The mixture was stirred at 80° C. for 1 hour. The reaction mixture was concentrated under reduced pressure to remove EtOH. The residue was purified by prep-HPLC (column: Phenomenex luna C18 150*25 mm*10 μm; mobile phase: [water(FA)-ACN]; B %: 10%-40%, 10 min) to afford Cpd. 9 (2.99 mg, 0.0047 mmol, 9.13%) was obtained as an orange solid. ¹H NMR (400 MHz, DMSO-d₆) δ=12.45-11.88 (m, 1H), 11.51-11.09 (m, 2H), 8.37 (s, 1H), 8.14-7.98 (m, 1H), 7.79-7.51 (m, 1H), 7.49-7.23 (m, 2H), 6.39-6.22 (m, 1H), 4.83-4.70 (m, 1H), 4.47-4.33 (m, 2H), 3.76 (d, J=1.2 Hz, 2H), 2.84-2.78 (m, 3H), 2.43-2.39 (m, 3H), 2.29 (d, J=5.2 Hz, 3H), 1.29-1.16 (m, 3H). LCMS m/z 466.1 (M+1).

### Example 12: Preparation of [3a(4)Z,10R]-6,9,10,15-tetramethyl-3,8-dioxo-3,5,8,9,10,11,13,15-octahydro-2H-1,17-(ethanediylidene)pyrazolo[4,3-m]dipyrrolo[3,2-f:3′,4′-i][1,4]oxazacyclopentadecine-20-carbonitrile (Cpd. 10)

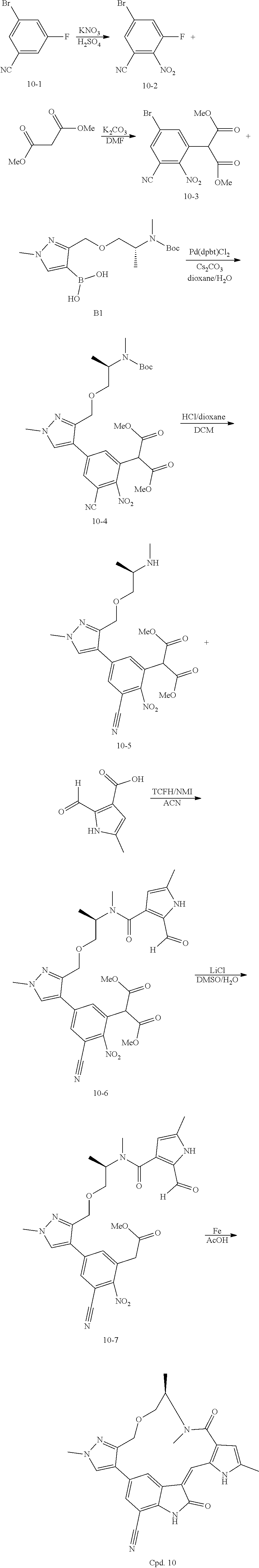

Step 1. To a solution of 3-bromo-5-fluoro-benzonitrile (3.00 g, 15.0 mmol, 1 eq) in H₂SO₄ (20 mL) was added KNO₃ (3.03 g, 30.0 mmol, 2.0 eq) at 0° C. The mixture was quenched with water (20 mL) at 0° C. and extracted with ethyl acetate (25 mL×3), the combined organic phase was dried over anhydrous sodium sulfate, filtered and concentrated to give a residue. The residue was purified by column chromatography (SiO₂, Petroleum ether/Ethyl acetate=10/1 to 2/1) to give 5-bromo-3-fluoro-2-nitro-benzonitrile (130 mg, 0.53 mmol, 3.54% yield) as a yellow solid. ¹H NMR (400 MHz, DMSO-d₆) δ=8.44 (dd, J=2.0, 10.4 Hz, 1H), 8.39 (t, J=1.8 Hz, 1H).

Step 2. To a solution of 5-bromo-3-fluoro-2-nitro-benzonitrile (1.30 g, 5.31 mmol, 1.0 eq), dimethyl propanedioate (841 mg, 6.37 mmol, 1.2 eq) in DMF (15 mL) was added K₂CO₃ (1.47 g, 10.6 mmol, 2 eq). The mixture was stirred at 80° C. for 17 hr. The mixture was quenched with water (20 mL) and extracted with ethyl acetate (25 mL×3), the combined organic phase was dried over anhydrous sodium sulfate, filtered and concentrated to give a residue. The residue was purified by column chromatography (SiO₂, Petroleum ether/Ethyl acetate=20/1 to 2/1) to give dimethyl 2-(5-bromo-3-cyano-2-nitro-phenyl)propanedioate (1.05 g, 2.94 mmol, 55.4% yield) as a colorless oil. ¹H NMR (400 MHz, DMSO-d₆) δ=8.57 (d, J=2.0 Hz, 1H), 8.21 (d, J=2.0 Hz, 1H), 5.53 (s, 1H), 3.69 (s, 6H).

Step 3. The mixture of dimethyl 2-(5-bromo-3-cyano-2-nitro-phenyl)propanedioate (2.00 g, 5.60 mmol, 1.0 eq), [3-[[(2R)-2-[tert-butoxycarbonyl(methyl)amino]propoxy]methyl]-1-methyl-pyrazol-4-yl]boronic acid (9.16 g, 28.0 mmol, 5.0 eq), ditert-butyl (cyclopentyl) phosphane; dichloropalladium; iron (365 mg, 0.56 mmol, 0.1 eq), K₂CO₃ (2.32 g, 16.8 mmol, 3.0 eq) in dioxane (40 mL) and H₂O (10 mL) was degassed and purged with N₂ for 3 times, and then the mixture was stirred at 70° C. for 3 h under N₂ atmosphere. The mixture was dried over anhydrous sodium sulfate, filtered and concentrated to give a residue. The residue was purified by column chromatography (SiO₂, Petroleum ether/Ethyl acetate=5/1 to 0/1) to give dimethyl 2-[5-[3-[[(2R)-2-[tert-butoxycarbonyl(methyl)amino]propoxy]methyl]-1-methyl-pyrazol-4-yl]-3-cyano-2-nitro-phenyl]propanedioate (1.72 g, 3.07 mmol, 54.9% yield) as an orange oil. ¹H NMR (400 MHz, DMSO-d₆) δ=8.43 (d, J=2.0 Hz, 1H), 8.13 (s, 1H), 7.61 (d, J=1.6 Hz, 1H), 5.60 (s, 1H), 4.46-4.41 (m, 2H), 4.38-4.32 (m, 2H), 3.92 (s, 3H), 3.79 (s, 3H), 3.72 (s, 6H), 2.63 (d, J=12.8 Hz, 3H), 1.37 (s, 9H).

Step 4. To a solution of dimethyl 2-[5-[3-[[(2R)-2-[tert-butoxycarbonyl(methyl)amino]propoxy]methyl]-1-methyl-pyrazol-4-yl]-3-cyano-2-nitro-phenyl]propanedioate (80.0 mg, 0.143 mmol, 1.0 eq) in DCM (4 mL) was added HCl/dioxane (4 M, 0.036 mL, 1.0 eq). The mixture was stirred at 25° C. for 1 h. The mixture was concentrated to give dimethyl 2-[3-cyano-5-[1-methyl-3-[[(2R)-2-(methylamino)propoxy]methyl]pyrazol-4-yl]-2-nitro-phenyl]propanedioate (80 mg, crude) as a brown oil. LCMS m/z 460.3 (M+1).

Step 5. To a solution of dimethyl 2-[3-cyano-5-[1-methyl-3-[[(2R)-2-(methylamino)propoxy]methyl]pyrazol-4-yl]-2-nitro-phenyl]propanedioate (100 mg, 0.217 mmol, 1.0 eq) and 2-formyl-5-methyl-1H-pyrrole-3-carboxylic acid (40.0 mg, 0.261 mmol, 1.2 eq) in ACN (10 mL) was added 1-methylimidazole (179 mg, 2.18 mmol, 10.0 eq) and [chloro(dimethylamino)methylene]-dimethyl-ammonium; hexafluorophosphate (91.6 mg, 0.326 mmol, 1.5 eq). The mixture was stirred at 20° C. for 1 hr. The mixture was concentrated to give a residue. The residue was purified by column chromatography (SiO₂, DCM/MeOH=1/0 to 10/1) to give dimethyl 2-[3-cyano-5-[3-[[(2R)-2-[(2-formyl-5-methyl-1H-pyrrole-3-carbonyl)-methyl-amino]propoxy]methyl]-1-methyl-pyrazol-4-yl]-2-nitro-phenyl]propanedioate (60.0 mg, 0.087 mmol, 40.0% yield) as a red solid. LCMS m/z 595.1 (M+1).

Step 6. To a solution of dimethyl 2-[3-cyano-5-[3-[[(2R)-2-[(2-formyl-5-methyl-1H-pyrrole-3-carbonyl)-methyl-amino]propoxy]methyl]-1-methyl-pyrazol-4-yl]-2-nitro-phenyl]propanedioate (50.0 mg, 0.084 mmol, 1.0 eq) in DMSO (2 mL) and H₂O (0.4 mL) was added LiCl (17.8 mg, 0.42 mmol, 5.0 eq). The mixture was stirred at 90° C. for 20 h. The mixture was quenched with water (10 mL) and extracted with ethyl acetate (5 mL×3), the combined organic phase was dried over anhydrous sodium sulfate, filtered and concentrated to give methyl 2-[3-cyano-5-[3-[[(2R)-2-[(2-formyl-5-methyl-1H-pyrrole-3-carbonyl)-methyl-amino]propoxy]methyl]-1-methyl-pyrazol-4-yl]-2-nitro-phenyl]acetate (80 mg, crude) as a colorless oil. LCMS m/z 537.1 (M+1).

Step 7. To a solution of methyl 2-[3-cyano-5-[3-[[(2R)-2-[(2-formyl-5-methyl-1H-pyrrole-3-carbonyl)-methyl-amino]propoxy]methyl]-1-methyl-pyrazol-4-yl]-2-nitro-phenyl]acetate (80.0 mg, 0.149 mmol, 1.0 eq) in AcOH (2 mL) was added Fe (41.6 mg, 0.746 mmol, 5.0 eq). The mixture was stirred at 90° C. for 1 hr. The mixture was filtered and concentrated to give a residue. The residue was purified by prep-HPLC (TFA condition; column: Welch Xtimate C18 150*25 mm*5 μm; mobile phase: [water(TFA)-ACN]; B %: 21%-51%, 10 min) to give Cpd. 10 (2.25 mg, 2.55% yield) as an orange solid. ¹H NMR (400 MHz, CDCl₃) δ=11.28 (d, J=4.0 Hz, 1H), 8.49 (s, 1H), 7.66 (s, 1H), 7.56 (s, 1H), 7.35 (d, J=2.0 Hz, 1H), 6.36 (d, J=2.4 Hz, 1H), 5.38-5.33 (m, 1H), 5.09-4.96 (m, 1H), 4.55-4.51 (m, 2H), 3.96 (s, 3H), 3.95-3.93 (m, 1H), 3.84-3.77 (m, 1H), 2.90 (s, 3H), 2.46 (s, 3H), 1.26 (s, 3H). LCMS m/z 457.2 (M+1).

### Example 13

Compounds 11-13 were prepared following General Method A with corresponding bromo pyrazole analog A2a or A4, boronic ester D1 or commercially available oxindole boronic ester 7-chloro-5-(4,4,5,5-tetramethyl-1,3,2-dioxaborolan-2-yl)indolin-2-one, and 2-formyl-5-methyl-1H-pyrrole-3-carboxylic acid.

| Cpd # | Structure | ¹H NMR (400 MHz, DMSO-d₆) δ ppm | MS m/z |
|---|---|---|---|
| 11 | 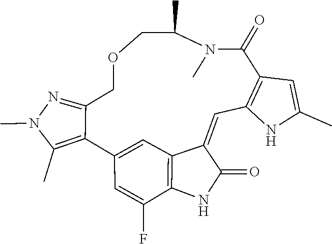 | 11.53 (s, 1H), 11.24 (s, 1H), 7.88 (s, J = 1.2 Hz, 1H), 7.42 (s, 1H), 7.02 (dd, J = 1.2 Hz, 1H), 6.27 (d, J = 2.0 Hz, 1H), 4.67-4.56 (m, 1H), 4.40-4.34 (m, 1H), 4.32-4.26 (m, 1H), 3.78-3.71 (m, 5H), 2.74-2.71 (m, 3H), 2.41-2.39 (m, 3H), 2.38-2.35 (m, 3H), 1.15 (d, J = 6.8 Hz, 3H) | 463.9 (M + 1)⁺ |
| 12 | 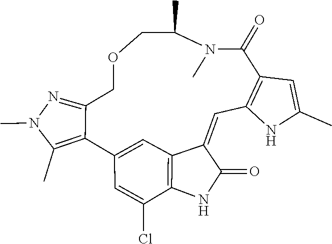 | 11.46 (s, 1H), 11.22 (s, 1H), 7.96 (s, 1H), 7.41(s, 1H), 7.15 (s, 1H), 6.28 (s, 1H), 4.68-4.54 (m, 1H), 4.40-4.25 (m, 2H), 3.82-3.67 (m, 5H), 2.72 (s, 3H), 2.49 (s, 3H), 2.41 (s, 3H), 1.19-1.10 (m, 3H) | 479.9 (M + 1)⁺ |
| 13 | 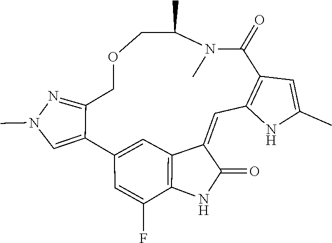 | 11.42 (s, 1H), 11.23 (s, 1H), 8.04-7.96 (m, 2H), 7.49-7.41 (m, 1H), 7.18-7.07 (m, 1H), 6.34-6.23 (m, 1H), 4.81-4.64 (m, 1H), 4.51-4.35 (m, 2H), 3.88-3.73 (m, 5H), 2.82-2.74 (m, 3H), 2.43-2.36 (m, 3H), 1.23 (s, 3H | 450.2 (M + 1)⁺ |

### Biochemical Assay

### Example 14

Kinase binding assays were performed at Eurofins/DiscoveRx using the general KINOMEscan Protocol (Fabian, M. A. et al., “A small molecule-kinase interaction map for clinical kinase inhibitors,” Nat. Biotechnol. 2005, 23(3):329-36). For most assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR. Results for compounds tested in this assay at a given concentration are reported as “% Ctrl”, where lower numbers indicate stronger binding in the matrix. % Ctrl was calculated as (test compound signal-positive control signal)/(negative control signal-positive control signal)×100. Dissociation constants (Kds) for test compound-kinase interactions were calculated by measuring the amount of kinase captured on the solid support as a function of the test compound concentration.

TABLE 1
Tyrosine Kinase Selectivity Profile of Compound
1 to Compound 5 (% Ctrl at 1 μM)

| | Tyrosine Kinase Target | | | | |
|---|---|---|---|---|---|
| | Cpd. 1 | Cpd. 2 | Cpd. 3 | Cpd. 4 | Cpd. 5 |
| | at 1 μM | at 1 μM | at 1 μM | at 1 μM | at 1 μM |
| DiscoveRx Gene Symbol | % Ctrl | % Ctrl | % Ctrl | % Ctrl | % Ctrl |
| ABL1(E255K)-phosphorylated | 92 | 45 | 100 | 94 | 79 |
| ABL1(F317I)-nonphosphorylated | 91 | 100 | 100 | 95 | 90 |
| ABL1(F3171)-phosphorylated | 64 | 98 | 97 | 98 | 97 |
| ABL1(F317L)-nonphosphorylated | 83 | 87 | 95 | 96 | 86 |
| ABL1(F317L)-phosphorylated | 86 | 78 | 93 | 90 | 83 |
| ABL1(H396P)-nonphosphorylated | 87 | 35 | 100 | 82 | 98 |
| ABL1(H396P)-phosphorylated | 96 | 64 | 100 | 96 | 80 |
| ABL1(M351T)-phosphorylated | 100 | 57 | 99 | 94 | 94 |
| ABL1(Q252H)-nonphosphorylated | 87 | 57 | 92 | 86 | 79 |
| ABL1(Q252H)-phosphorylated | 99 | 62 | 100 | 89 | 89 |
| ABL1(T315I)-nonphosphorylated | 83 | 17 | 100 | 100 | 100 |
| ABL1(T315I)-phosphorylated | 95 | 5.3 | 69 | 35 | 70 |
| ABL1(Y253F)-phosphorylated | 92 | 60 | 91 | 79 | 73 |
| ABL1-nonphosphorylated | 87 | 55 | 92 | 84 | 87 |
| ABL1-phosphorylated | 81 | 50 | 96 | 90 | 79 |
| ABL2 | 87 | 96 | 97 | 97 | 96 |
| ALK | 87 | 100 | 100 | 100 | 71 |
| ALK(C1156Y) | 100 | 93 | 89 | 100 | 90 |
| ALK(L1196M) | 89 | 94 | 94 | 94 | 86 |
| AXL | 85 | 43 | 86 | 67 | 86 |
| BLK | 87 | 98 | 92 | 100 | 87 |
| BMX | 67 | 95 | 91 | 93 | 100 |
| BRK | 92 | 100 | 95 | 83 | 79 |
| BTK | 94 | 100 | 86 | 78 | 68 |
| CSF1R | 98 | 82 | 100 | 92 | 96 |
| CSF1R-autoinhibited | 82 | 100 | 71 | 75 | 75 |
| CSK | 97 | 100 | 94 | 100 | 96 |
| CTK | 58 | 93 | 80 | 76 | 84 |
| DDR1 | 97 | 100 | 98 | 20 | 93 |
| DDR2 | 95 | 100 | 100 | 79 | 76 |
| EGFR | 100 | 66 | 79 | 82 | 96 |
| EGFR(E746-A750del) | 95 | 100 | 98 | 96 | 95 |
| EGFR(G719C) | 81 | 80 | 100 | 93 | 100 |
| EGFR(G719S) | 80 | 77 | 100 | 96 | 98 |
| EGFR(L747-E749del, A750P) | 100 | 85 | 90 | 75 | 74 |
| EGFR(L747-S752del, P753S) | 100 | 100 | 90 | 90 | 86 |
| EGFR(L747-T751del, Sins) | 41 | 54 | 95 | 100 | 100 |
| EGFR(L858R) | 100 | 78 | 96 | 95 | 90 |
| EGFR(L858R, T790M) | 89 | 62 | 72 | 77 | 64 |
| EGFR(L861Q) | 82 | 62 | 98 | 89 | 94 |
| EGFR(S752-1759del) | 88 | 80 | 94 | 93 | 100 |
| EGFR(T790M) | 88 | 85 | 87 | 90 | 80 |
| EPHA1 | 100 | 100 | 85 | 71 | 82 |
| EPHA2 | 97 | 85 | 93 | 96 | 100 |
| EPHA3 | 86 | 99 | 75 | 83 | 79 |
| EPHA4 | 96 | 100 | 92 | 92 | 93 |
| EPHA5 | 93 | 99 | 100 | 100 | 97 |
| EPHA6 | 94 | 100 | 93 | 97 | 91 |
| EPHA7 | 91 | 89 | 100 | 100 | 98 |
| EPHA8 | 100 | 92 | 90 | 82 | 77 |
| EPHB1 | 87 | 99 | 100 | 97 | 92 |
| EPHB2 | 97 | 97 | 99 | 96 | 90 |
| EPHB3 | 99 | 100 | 93 | 93 | 89 |
| EPHB4 | 86 | 100 | 97 | 98 | 91 |
| EPHB6 | 100 | 93 | 86 | 86 | 92 |
| ERBB2 | 98 | 100 | 100 | 100 | 100 |
| ERBB3 | 100 | 100 | 74 | 76 | 71 |
| ERBB4 | 95 | 67 | 96 | 100 | 97 |
| FAK | 85 | 96 | 100 | 96 | 96 |
| FER | 97 | 100 | 73 | 68 | 68 |
| FES | 92 | 100 | 99 | 100 | 89 |
| FGFR1 | 100 | 100 | 80 | 75 | 84 |
| FGFR2 | 96 | 85 | 90 | 99 | 100 |
| FGFR3 | 96 | 88 | 89 | 87 | 90 |
| FGFR3(G697C) | 100 | 93 | 89 | 79 | 93 |
| FGFR4 | 95 | 100 | 92 | 88 | 88 |
| FGR | 91 | 100 | 98 | 100 | 93 |
| FLT1 | 97 | 79 | 92 | 92 | 93 |
| FLT3 | 92 | 67 | 57 | 100 | 94 |
| FLT3(D835H) | 96 | 31 | 100 | 65 | 79 |
| FLT3(D835V) | 61 | 7.9 | 100 | 75 | 89 |
| FLT3(D835Y) | 97 | 36 | 81 | 96 | 100 |
| FLT3(ITD) | 96 | 65 | 97 | 87 | 91 |
| FLT3(ITD, D835V) | 94 | 21 | 96 | 77 | 76 |
| FLT3(ITD, F691L) | 90 | 21 | 73 | 36 | 75 |
| FLT3(K663Q) | 80 | 47 | 100 | 90 | 87 |
| FLT3(N841I) | 97 | 24 | 90 | 78 | 86 |
| FLT3(R834Q) | 97 | 50 | 100 | 100 | 98 |
| FLT3-autoinhibited | 92 | 83 | 93 | 93 | 87 |
| FLT4 | 94 | 19 | 100 | 100 | 98 |
| FRK | 93 | 98 | 97 | 78 | 80 |
| FYN | 91 | 100 | 100 | 93 | 77 |
| HCK | 93 | 100 | 100 | 100 | 100 |
| IGF1R | 100 | 88 | 95 | 94 | 98 |
| INSR | 95 | 100 | 78 | 81 | 94 |
| INSRR | 94 | 100 | 99 | 91 | 99 |
| ITK | 89 | 80 | 100 | 99 | 96 |
| JAK1(JH1domain-catalytic) | 82 | 100 | 100 | 94 | 100 |
| JAK1(JH2domain-pseudokinase) | 100 | 30 | 3.1 | 0.25 | 0.85 |
| JAK2(JH1domain-catalytic) | 98 | 91 | 93 | 57 | 89 |
| JAK3(JH1domain-catalytic) | 81 | 49 | 70 | 12 | 79 |
| KIT | 91 | 100 | 100 | 100 | 98 |
| KIT(A829P) | 93 | 92 | 99 | 92 | 84 |
| KIT(D816H) | 74 | 98 | 94 | 80 | 77 |
| KIT(D816V) | 97 | 60 | 97 | 100 | 100 |
| KIT(L576P) | 88 | 100 | 100 | 100 | 97 |
| KIT(V559D) | 91 | 98 | 100 | 100 | 96 |
| KIT(V559D, T670I) | 93 | 82 | 100 | 94 | 95 |
| KIT(V559D, V654A) | 100 | 92 | 100 | 99 | 91 |
| KIT-autoinhibited | 81 | 100 | 87 | 82 | 83 |
| LCK | 95 | 100 | 87 | 88 | 94 |
| LTK | 74 | 100 | 100 | 100 | 93 |
| LYN | 100 | 74 | 90 | 87 | 91 |
| MERTK | 78 | 100 | 98 | 48 | 86 |
| MET | 84 | 95 | 100 | 98 | 96 |
| MET(M1250T) | 83 | 98 | 100 | 100 | 100 |
| MET(Y1235D) | 98 | 87 | 100 | 100 | 100 |
| MST1R | 100 | 100 | 98 | 100 | 95 |
| MUSK | 93 | 100 | 98 | 98 | 100 |
| PDGFRA | 87 | 73 | 92 | 96 | 95 |
| PDGFRB | 100 | 79 | 100 | 100 | 100 |
| PYK2 | 100 | 100 | 94 | 92 | 83 |
| RET | 94 | 90 | 96 | 100 | 95 |
| RET(M918T) | 84 | 92 | 100 | 98 | 87 |
| RET(V804L) | 82 | 80 | 98 | 95 | 84 |
| RET(V804M) | 100 | 77 | 100 | 90 | 90 |
| ROS1 | 91 | 97 | 62 | 68 | 69 |
| SRC | 89 | 100 | 100 | 100 | 100 |
| SRMS | 93 | 90 | 69 | 68 | 61 |
| SYK | 98 | 100 | 68 | 64 | 90 |
| TEC | 91 | 83 | 96 | 99 | 94 |
| TIE1 | 100 | 61 | 93 | 84 | 91 |
| TIE2 | 95 | 63 | 97 | 91 | 92 |
| TNK1 | 100 | 92 | 94 | 94 | 91 |
| TNK2 | 100 | 89 | 97 | 100 | 87 |
| TRKA | 67 | 20 | 61 | 59 | 53 |
| TRKB | 87 | 12 | 100 | 100 | 100 |
| TRKC | 94 | 22 | 100 | 100 | 98 |
| TXK | 100 | 100 | 91 | 78 | 94 |
| TYK2(JH1domain-catalytic) | 31 | 100 | 71 | 48 | 69 |
| TYK2(JH2domain-pseudokinase) | 1.5 | 0.6 | 0 | 0.2 | 0 |
| TYRO3 | 100 | 100 | 94 | 97 | 84 |
| VEGFR2 | 93 | 48 | 92 | 94 | 92 |
| YES | 100 | 100 | 99 | 95 | 86 |
| ZAP70 | 94 | 96 | 55 | 56 | 63 |

TABLE 2
Dissociation constants (Kds) against JAK Family

| | JAK1(JH1 | JAK1(JH2 | JAK2(JH1 | JAK3(JH1 | TYK2(JH1 | TYK2(JH2 |
|---|---|---|---|---|---|---|
| | domain- | domain- | domain- | domain- | domain- | domain- |
| Cpd | catalytic) | pseudokinase) | catalytic) | catalytic) | catalytic) | pseudokinase) |
| # | K_{d} (nM) | K_{d} (nM) | K_{d} (nM) | K_{d} (nM) | K_{d} (nM) | K_{d} (nM) |
| 1 | >10000 | | >10000 | >10000 | >10000 | 220 |
| 2 | >10000 | | 2300 | 510 | >10000 | 1.2 |
| 3 | >10000 | 120 | >10000 | 7700 | >10000 | 3 |
| 4 | >10000 | 29 | 1800 | 59 | 1500 | 1.9 |
| 5 | >10000 | 50 | >10000 | >10000 | >10000 | 0.18 |
| 6 | >10000 | 160 | >10000 | >10000 | >10000 | 2.5 |
| 7 | >10000 | 47 | >10000 | >10000 | >10000 | 0.51 |
| 8 | >10000 | | >10000 | >10000 | >10000 | 170 |
| 9 | >10000 | 130 | >10000 | 3100 | >10000 | 1 |
| 10 | >1000 | | >1000 | >1000 | | 46 |
| 11 | >10000 | 730 | >10000 | 5200 | >10000 | 3.4 |
| 12 | >10000 | 120 | >10000 | >10000 | >10000 | 1.3 |
| 13 | >10000 | 410 | >10000 | >10000 | >10000 | 1.0 |

### Example 15: Phosphorylation of STATS in IFNa-stimulated CD3+ T Cells

Inhibition of the phosphorylation of STATS in IFNa-stimulated CD3+ T cells were evaluated at HD Bioscience, Inc. (6122 Nancy Ridge Drive, San Diego, CA 92121).

PBMC isolation: Human whole blood was obtained from San Diego Blood Bank in a tube containing heparin. Peripheral blood mononuclear cells (PBMCs) were isolated by density gradient centrifugation with lymphoprep. Briefly, 15 mL Lymphoprep buffer was added to the bottom of the SepMate tube. The tube was spined down shortly. 8 ml blood was diluted using Robosep buffer. Blood/Robosep buffer mixture was added into the tube and was centrifuged at 1200×g for 10 min at room temperature. The pellet was washed with Robosep buffer by centrifuge 300×g for 8 min at room temperature. The pellet was re-suspended in 10 mL 1×RBC lysis buffer and incubated for 10 min at room temperature. 20 mL Robosep buffer was added to stop the reaction. The pellet was spined down and resuspended in 2 mL Robosep buffer.

PBMC plating and IFNa treatment: PBMC cells were resuspended at a concentration of 1.25e6 cells/mL. CD3 antibodies were added at a ratio of 1 μL antibody per 90 μL cell suspension. 80 μL/well (1e5 cells) was plated in the 96 well plate. The cells were treated with indicated compounds and were incubated at 37° C. for 30 minutes. 20 μL of IFNa (PBL, Cat #11101) was added to the cells and the cells were incubated at 37° C. for 30 minutes.

Fixation, permeabilization, and intracellular staining: The PBMC cells were immediately fixed with 500 μL (per well) of pre-warmed 1× Lyse/Fix Buffer at 37° C. for 10 minutes following stimulation. The cells were washed with 300 μL FACS staining buffer with centrifuge at 500×g for 5 minutes. The cells were thoroughly resuspended in 500 μL of BD Phosflow Perm Buffer III. Allow cells to permeabilize for 30 minutes at 4° C. in the dark. The cells were further washed with centrifuge at 500×g for 5 minutes with 500 μL 1× Permeabilization buffer and were resuspended in 100 μL 1× Permeabilization buffer containing 2.5 μL pSTAT5 antibody (BD Biosciences, Cat #562077). The cells were stained at room temperature for 1 hour, followed by centrifuge at 500×g for 5 minutes to remove the supernatant. The cells were washed with 300 μL FACS staining buffer with centrifuge and were resuspended cells in 300p L of FACS buffer. pSTAT5 expression was analyzed via flow cytometry. Results are shown in Table 3.

TABLE 3

| | Cpd. 2 | Cpd. 5 | Cpd. 7 |
|---|---|---|---|
| IC₅₀ (nM) | 146.8 | 15.7 | 48.9 |

What is claimed is:

1. A compound of the formula I, or a pharmaceutically acceptable salt thereof, 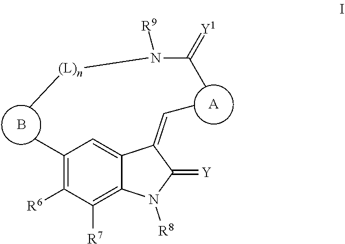
wherein
A is 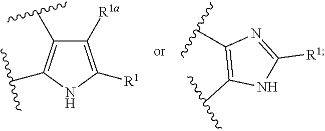
B is 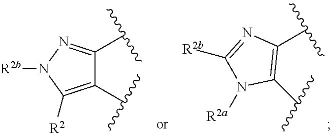
each L is independently —C(R³)(R⁴)—, —C(O)—, —O—, —N(R⁵)—, —S—, —S(O)— or —S(O)₂—, provided that (L)ₙ does not comprise a —O—O—, a —O—S—, a —S—S—, or a —O—N(R⁵)— bond, and (L)ₙ-N(R⁹)— does not comprise a —O—N(R⁹)— or a —S—N(R⁹)— bond;
Y and Y¹ are each independently O or S;
each of R¹, R^{1a}, and R² is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R^{2a}, R⁵, R⁸, and R⁹ is independently H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R^{2b} is C₁-C₄ alkyl, C₂-C₄ alkenyl, C₂-C₄ alkynyl, C₃-C₄ cycloalkyl, or 3- to 4-membered heterocycloalkyl, wherein each hydrogen atom in C₁-C₄ alkyl, C₂-C₄ alkenyl, C₂-C₄ alkynyl, C₃-C₄ cycloalkyl, and 3- to 4-membered heterocycloalkyl is independently optionally substituted by deuterium, halogen, —O(H or C₁-C₂ alkyl), —OC(O)C₁-C₂ alkyl, —OC(O)N(H or C₁-C₂ alkyl)₂, —OS(O)C₁-C₂ alkyl, —OS(O)₂C₁-C₂ alkyl, —OS(O)N(H or C₁-C₂ alkyl)₂, —OS(O)₂N(H or C₁-C₂ alkyl)₂, —S(H or C₁-C₂ alkyl), —S(O)C₁-C₂ alkyl, —S(O)₂C₁-C₂ alkyl, —S(O)N(H or C₁-C₂ alkyl)₂, —S(O)₂N(H or C₁-C₂ alkyl)₂, —N(H or C₁-C₂ alkyl)₂, —N(H or C₁-C₂ alkyl)C(O)C₁-C₂ alkyl, —N(H or C₁-C₂ alkyl)C(O)O(H or C₁-C₂ alkyl), —N(H or C₁-C₂ alkyl)C(O)N(H or C₁-C₂ alkyl)₂, —N(H or C₁-C₂ alkyl)S(O)C₁-C₂ alkyl, —N(H or C₁-C₂ alkyl)S(O)₂C₁-C₂ alkyl, —N(H or C₁-C₂ alkyl)S(O)N(H or C₁-C₂ alkyl)₂, —N(H or C₁-C₂ alkyl)S(O)₂N(H or C₁-C₂ alkyl)₂, —C(O)C₁-C₂ alkyl, —C(O)O(H or C₁-C₂ alkyl), —C(O)N(H or C₁-C₂ alkyl)₂, —P(H or C₁-C₂ alkyl)₂, —P(O)(H or C₁-C₂ alkyl)₂, —P(O)₂(H or C₁-C₂ alkyl)₂, —P(O)N(H or C₁-C₂ alkyl)₂, —P(O)₂N(H or C₁-C₂ alkyl)₂, —P(O)O(H or C₁-C₂ alkyl), —P(O)₂O(H or C₁-C₂ alkyl), —CN, or —NO₂;
each R³ and R⁴ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, and R⁵ taken together with the atoms to which they are attached form a C₃-C₆ cycloalkyl or a 4- to 8-membered heterocycloalkyl, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R⁶ and R⁷ is independently H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁶ and R⁷ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 4- to 8-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, C₁-C₆ alkyl-C₆-C₁₀ aryl, and 5- to 10-membered heteroaryl; and
n is 2, 3, 4, 5, 6, 7, or 8.

2. The compound of claim 1 having the formula II 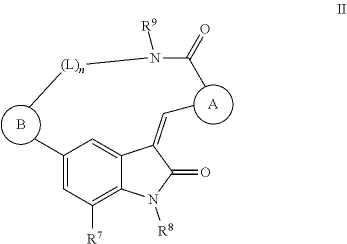
or a pharmaceutically acceptable salt thereof.

3. The compound of claim 1 having the formula III 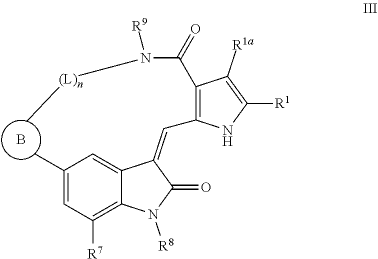
or a pharmaceutically acceptable salt thereof.

4. The compound of claim 1 or 2, having the formula IV 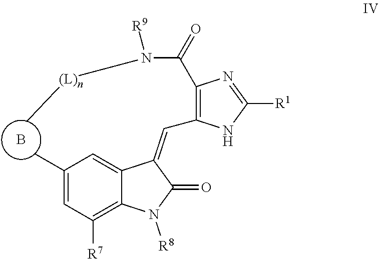
or a pharmaceutically acceptable salt thereof.

5. The compound of claim 1 or 2, having the formula V 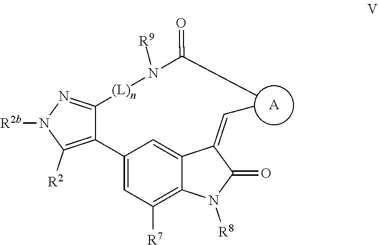
or a pharmaceutically acceptable salt thereof.

6. The compound of claim 1 or 2, having the formula VI 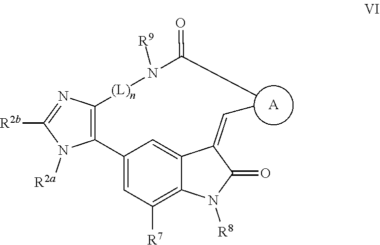
or a pharmaceutically acceptable salt thereof.

7. The compound of any one of claims 1 to 3, having the formula VII 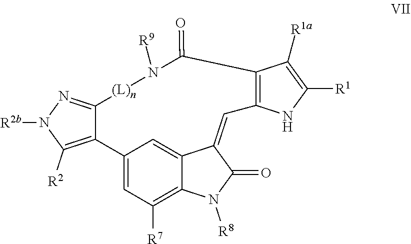
or a pharmaceutically acceptable salt thereof.

8. The compound of claim 1 or 2, having the formula VIII 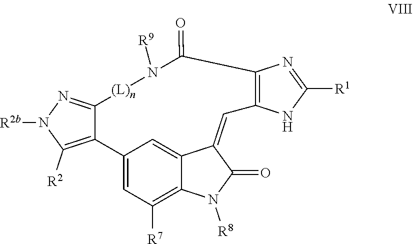
or a pharmaceutically acceptable salt thereof.

9. The compound of any one of claims 1 to 3, having the formula IX 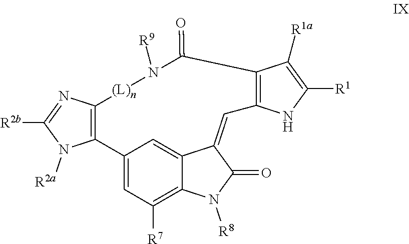
or a pharmaceutically acceptable salt thereof.

10. The compound of claim 1 or 2, having the formula X 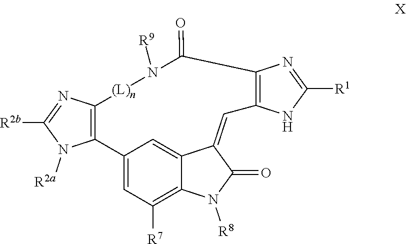
or a pharmaceutically acceptable salt thereof.

11. The compound of any one of claims 1 to 3, having the formula XI 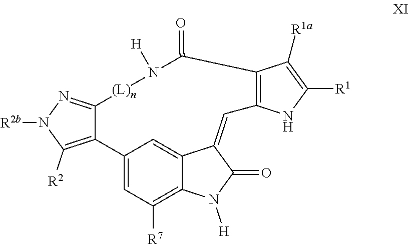
or a pharmaceutically acceptable salt thereof.

12. The compound of any one of claims 1 to 3, having the formula XII 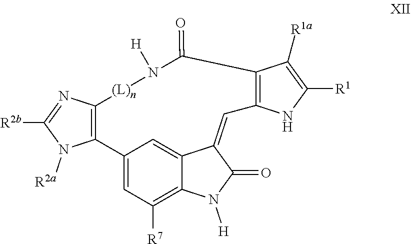
or a pharmaceutically acceptable salt thereof.

13. The compound of any one of the preceding claims, or a pharmaceutically acceptable salt thereof, wherein R¹, when present, is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or or R¹ is H, —CN or methyl.

14. The compound of any one of claims 1 to 3, 5 to 7, 9, 11, 12, or 13, or a pharmaceutically acceptable salt thereof, wherein R^{1a}, when present, is H, —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂, or R^{1a} is H, —CN or methyl.

15. The compound of any one of claims 1 to 5, 7, 8, 11, 13, or 14, or a pharmaceutically acceptable salt thereof, wherein R², when present, is H, deuterium, halogen, C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, 5- to 10-membered heteroaryl, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 4- to 8-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R², when present, is H, —CN, or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R², when present, is H, —CN, or methyl.

16. The compound of any one of the claims 1 to 4, 6, 9, 10, or 12 to 15, or a pharmaceutically acceptable salt thereof, wherein R^{2a}, when present, is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R^{2a}, when present, is H or methyl.

17. The compound of any one of the preceding claims, or a pharmaceutically acceptable salt thereof, wherein R^{2b}, when present, is C₁-C₄ alkyl or C₃-C₄ cycloalkyl, wherein each hydrogen atom in C₁-C₆ alkyl and C₃-C₄ cycloalkyl is independently optionally substituted by deuterium or halogen; or R^{2b} is methyl, ethyl, isopropyl, or cyclopropyl.

18. The compound of any one of the preceding claims, or a pharmaceutically acceptable salt thereof, wherein n is 3.

19. The compound of any one of claims 1 to 17, or a pharmaceutically acceptable salt thereof, wherein n is 4.

20. The compound of any one of claims 1 to 17, or a pharmaceutically acceptable salt thereof, wherein n is 5.

21. The compound of any one of claims 1 to 17, or a pharmaceutically acceptable salt thereof, wherein n is 6.

22. The compound of any one of the preceding claims, or a pharmaceutically acceptable salt thereof, wherein each L is independently selected from the group consisting of —C(O)—, —O—, —CH₂—, —C(H)(CH₃)—, —C(H)(OH)—, —C(H)(C(O)OR^{c})—, —C(H)(C(O)NR^{c}R^{d})—, —NH—, and —NCH₃—; or each L is independently selected from the group consisting of —C(O)—, —O—, —CH₂—, —C(H)(CH₃)—, —C(H)(OH)—, —C(H)(C(O)OR^{c})—, and —C(H)(C(O)NR^{c}R^{d})—.

23. The compound of any one of claims 1 or 13 to 22, or a pharmaceutically acceptable salt thereof, wherein Y is O.

24. The compound of any one of claims 1 or 13 to 23, or a pharmaceutically acceptable salt thereof, wherein Y¹ is O.

25. The compound of any one of claims 1 or 13 to 25, or a pharmaceutically acceptable salt thereof, wherein R⁶, when present, is independently H, deuterium, fluoro, chloro, —CN, or methyl.

26. The compound of any one of the preceding claims, or a pharmaceutically acceptable salt thereof, wherein R⁷ is independently H, deuterium, fluoro, chloro, —CN, or methyl.

27. The compound of any one of claims 1 to 10 or 13 to 26, or a pharmaceutically acceptable salt thereof, wherein R⁸ is H or methyl.

28. The compound of any one of claims 1 to 10 or 13 to 27, or a pharmaceutically acceptable salt thereof, wherein R⁹ is H or methyl.

29. The compound of any one of the preceding claims, or a pharmaceutically acceptable salt thereof, wherein -(L)ₙ- is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —CH₂O(CH₂)₃—, —CH₂O(C(CH₃)H)₂—, —(CH₂)₂O(CH₂)₂—, —CH₂OCH₂(C(CH₃)H)—, —(CH₂)₂OCH₂(C(CH₃)H)—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —O—(CH₂)₄—, —O—(CH₂)₂CH(CH₃)— —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N((CH₂)₂CH₃)—(CH₂)₂—, —CH₂N(CH(CH₃)₂)—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—; or —O(CH₂)₂—, —O(CH₂)₃—, —O(CH₂)₄—, —CH₂OCH₂(C(CH₃)H)—, —CH₂O(CH₂)₂—, or —CH₂O(CH₂)₃—; or —O(CH₂)₃—, —CH₂OCH₂(C(CH₃)H)—, or —CH₂O(CH₂)₂—.

30. The compound of claim 1, or a pharmaceutically acceptable salt thereof, selected from the group consisting of [3a(4)Z]-6,9,15,16-tetramethyl-9,10,11,12-tetrahydro-15H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,15,16-tetramethyl-10,11,13,15-tetrahydro-2H-1,17-(ethanediylidene)pyrazolo[4,3-m]dipyrrolo[3,2f:3′,4′-i][1,4]oxazacyclopentadecine-3,8(5H,9H)-dione;
[3a(4)Z,10R]-20-chloro-6,10,15-trimethyl-10,11,13,15-tetrahydro-2H-1,17-(ethanediylidene)pyrazolo[4,3-m]dipyrrolo[3,2 f:3′,4′-i][1,4]oxazacyclopentadecine-3,8(5H,9H)-dione;
[3a(4)Z,10R]-20-chloro-10,15-dimethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z,10S]-20-chloro-6,9,10,15-tetramethyl-10,11,13,15-tetrahydro-2H-1,17-(ethanediylidene)pyrazolo[4,3-m]dipyrrolo[3,2 f:3′,4′-i][1,4]oxazacyclopentadecine-3,8(5H,9H)-dione;
[3a(4)Z]-20-chloro-6,9,15-trimethyl-10,11,13,15-tetrahydro-2H-1,17-(ethanediylidene)pyrazolo[4,3-m]dipyrrolo[3,2f:3′,4′-i][1,4]oxazacyclopentadecine-3,8(5H,9H)-dione;
[3a(4)Z,10R]-20-chloro-6,9,10,15-tetramethyl-10,11,13,15-tetrahydro-2H-1,17-(ethanediylidene)imidazo[4,5-f]pyrazolo[4,3-m]pyrrolo[3,4-i][1,4]oxazacyclopentadecine-3,8(5H,9H)-dione;
[3a(4)Z,10R]-20-chloro-6,9,10,15-tetramethyl-10,11,13,16-tetrahydro-2H-1,17-(ethanediylidene)imidazo[4,5-m]dipyrrolo[3,2-f:3′,4′-i][1,4]oxazacyclopentadecine-3,8(5H,9H)-dione;
[3a(4)Z,10R]-6,9,10,15-tetramethyl-3,8-dioxo-3,5,8,9,10,11,13,15-octahydro-2H-1,17-(ethanediylidene)pyrazolo[4,3-m]dipyrrolo[3,2 f:3′,4′-i][1,4]oxazacyclopentadecine-20-carbonitrile;
[3a(4)Z,10R]-20-fluoro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione;
[3a(4)Z,10R]-20-chloro-6,9,10,15,16-pentamethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione; and
[3a(4)Z,10R]-20-fluoro-6,9,10,15-tetramethyl-10,11,13,15-tetrahydro-2H-1,17-ethenopyrazolo[4,3-m]dipyrrolo[3,2-f:3′,4′-i][1,4]oxazacyclopentadecine-3,8(5H,9H)-dione.

31. A pharmaceutical composition comprising at least one compound of any one of claims 1 to 30, or a pharmaceutically acceptable salt thereof, and optionally one or more pharmaceutically acceptable excipients.

32. A method of treating disease, such as autoimmune disease, comprising administering to a subject in need of such treatment an effective amount of a compound of any one of claims 1 to 30, or a pharmaceutically acceptable salt thereof.

33. A compound of any one of claims 1 to 30, or a pharmaceutically acceptable salt thereof, for use in a method of treating an autoimmune disease in a subject.

34. A compound of any one of claims 1 to 30, or a pharmaceutically acceptable salt thereof, for treating an autoimmune disease in a subject.

35. Use of a compound of any one of claims 1 to 30, or a pharmaceutically acceptable salt thereof, in the manufacture of a medicament for treating an autoimmune disease in a subject.
